SEQUENCE LISTING (48 sequences)

SEQ ID NO: 1 - protein, 21 aa, synthetic construct
  SITE 8: note = phosphorylation
  SITE 11: note = phosphorylation
  SITE 14: note = phosphorylation
GDRSGYSSPGSPGTPGSRSRT

SEQ ID NO: 2 - protein, 16 aa, synthetic construct
  SITE 4: note = phosphorylation
  SITE 12: note = phosphorylation
VYKSPVVSGDTSPRHL

SEQ ID NO: 3 - protein, 18 aa, synthetic construct
  SITE 11: note = phosphorylation
  SITE 16: note = phosphorylation
SSTGSIDMVDSPQLATLA

SEQ ID NO: 4 - protein, 16 aa, synthetic construct
VYKSPVVSGDTSPRHL

SEQ ID NO: 5 - protein, 30 aa, synthetic construct
  SITE 18: note = phosphorylation
  SITE 26: note = phosphorylation
RENAKAKTDHGAEIVYKSPVVSGDTSPRHL

SEQ ID NO: 6 - protein, 16 aa, synthetic construct
  SITE 14: note = phosphorylation
RQEFEVMEDHAGTYGL

SEQ ID NO: 7 - protein, 16 aa, synthetic construct
  SITE 7: note = phosphorylation
  SITE 9: note = phosphorylation
PGSRSRTPSLPTPPTR

SEQ ID NO: 8 - protein, 16 aa, synthetic construct
  SITE 7: note = phosphorylation
  SITE 10: note = phosphorylation
GYSSPGSPGTPGSRSR

SEQ ID NO: 9 - protein, 18 aa, synthetic construct
  SITE 4: note = phosphorylation
  SITE 9: note = phosphorylation
GDTSPRHLSNVSSTGSID

SEQ ID NO: 10 - protein, 17 aa, synthetic construct
  SITE 3: note = phosphorylation
  SITE 6: note = phosphorylation
  SITE 13: note = phosphorylation
  SITE 15: note = phosphorylation
PGSPGTPGSRSRTPSLP

SEQ ID NO: 11 - protein, 12 aa, synthetic construct
  SITE 3: note = phosphorylation
HLSNVSSTGSID

SEQ ID NO: 12 - protein, 10 aa, synthetic construct
  SITE 6: note = phosphorylation
VSGDTSPRHL

SEQ ID NO: 13 - protein, 55 aa, synthetic construct
  MOD_RES 55: note = C-terminal amide
AKFVAAWTLKAAAVVRQYIKANSKFIGITELVVRFNNFTVSFWLRVPKVSASHLE

SEQ ID NO: 14 - protein, 55 aa, synthetic construct
  MOD_RES 55: note = C-terminal amide
  LIPID 54: note = palmitoylated
  LIPID 55: note = palmitoylated
AKFVAAWTLKAAAGSQYIKANSKFIGITELGSFNNFTVSFWLRVPKVSASHLEKK

SEQ ID NO: 15 - protein, 75 aa, synthetic construct
  MOD_RES 75: note = C-terminal amide
AKFVAAWTLKAAAGSQYIKANSKFIGITELGSFNNFTVSFWLRVPKVSASHLEGSLINSTKIYSYFPSVISKVNQ

SEQ ID NO: 16 - protein, 53 aa, synthetic construct
  MOD_RES 53: note = C-terminal amide
AKFVAAWTLKAAARRQYIKANSKFIGITELRRFNNFTVSFWLRVPKVSASHLE

SEQ ID NO: 17 - protein, 53 aa, synthetic construct
  MOD_RES 53: note = C-terminal amide
AKFVAAWTLKAAARKQYIKANSKFIGITELRKFNNFTVSFWLRVPKVSASHLE

SEQ ID NO: 18 - DNA, 24 bp, synthetic construct
  source 1..24: mol_type = other DNA
  modified_base 1..24: mod_base = OTHER; note = phosphorothioate (ps) internucleotide linkages
tcgtcgttttgtcgttttgtcgtt

SEQ ID NO: 19 - DNA, 22 bp, synthetic construct
  source 1..22: mol_type = other DNA
  modified_base 1..22: mod_base = OTHER; note = phosphorothioate (ps) internucleotide linkages
tgactgtgaacgttcgagatga

SEQ ID NO: 20 - DNA, 22 bp, synthetic construct
  source 1..22: mol_type = other DNA
  modified_base 1..22: mod_base = OTHER; note = phosphorothioate (ps) internucleotide linkages
tcgtcgttttcggcgcgcgccg

SEQ ID NO: 21 - DNA, 20 bp, synthetic construct
  source 1..20: mol_type = other DNA
  modified_base 1..2: mod_base = OTHER; note = phosphorothioate (ps) internucleotide linkages
  modified_base 3..14: mod_base = OTHER; note = phosphodiester (po) linkages
  modified_base 15..20: mod_base = OTHER; note = phosphorothioate (ps) internucleotide
gggggacgatcgtcgggggg

SEQ ID NO: 22 - DNA, 21 bp, synthetic construct
  source 1..21: mol_type = other DNA
  modified_base 1..3: mod_base = OTHER; note = phosphorothioate (ps) internucleotide
  modified_base 16..21: mod_base = OTHER; note = phosphorothioate (ps) internucleotide
  modified_base 4..15: mod_base = OTHER; note = phosphodiester (po) linkages
ggggacgacgtcgtggggggg

SEQ ID NO: 23 - protein, 13 aa, synthetic construct
AKFVAAWTLKAAA

SEQ ID NO: 24 - protein, 15 aa, synthetic construct
QYIKANSKFIGITEL

SEQ ID NO: 25 - protein, 21 aa, synthetic construct
FNNFTVSFWLRVPKVSASHLE

SEQ ID NO: 26 - protein, 20 aa, synthetic construct
LINSTKIYSYFPSVISKVNQ

SEQ ID NO: 27 - protein, 25 aa, synthetic construct
  LIPID 1: note = palmitoylated
  LIPID 2: note = palmitoylated
  LIPID 24: note = palmitoylated
  LIPID 25: note = palmitoylated
  SITE 10: note = phosphorylation
  SITE 13: note = phosphorylation
  SITE 16: note = phosphorylation
KKGDRSGYSSPGSPGTPGSRSRTKK

SEQ ID NO: 28 - protein, 20 aa, synthetic construct
  LIPID 1: note = palmitoylated
  LIPID 2: note = palmitoylated
  LIPID 19: note = palmitoylated
  LIPID 20: note = palmitoylated
  SITE 6: note = phosphorylation
  SITE 14: note = phosphorylation
KKVYKSPVVSGDTSPRHLKK

SEQ ID NO: 29 - protein, 22 aa, synthetic construct
  LIPID 1: note = palmitoylated
  LIPID 2: note = palmitoylated
  LIPID 21: note = palmitoylated
  LIPID 22: note = palmitoylated
  SITE 13: note = phosphorylation
  SITE 18: note = phosphorylation
KKSSTGSIDMVDSPQLATLAKK

SEQ ID NO: 30 - protein, 20 aa, synthetic construct
  LIPID 1: note = palmitoylated
  LIPID 2: note = palmitoylated
  LIPID 19: note = palmitoylated
  LIPID 20: note = palmitoylated
KKVYKSPVVSGDTSPRHLKK

SEQ ID NO: 31 - protein, 34 aa, synthetic construct
  LIPID 1: note = palmitoylated
  LIPID 2: note = palmitoylated
  LIPID 33: note = palmitoylated
  LIPID 34: note = palmitoylated
  SITE 20: note = phosphorylation
  SITE 28: note = phosphorylation
KKRENAKAKTDHGAEIVYKSPVVSGDTSPRHLKK

SEQ ID NO: 32 - protein, 20 aa, synthetic construct
  LIPID 1: note = palmitoylated
  LIPID 2: note = palmitoylated
  LIPID 19: note = palmitoylated
  LIPID 20: note = palmitoylated
  SITE 16: note = phosphorylation
KKRQEFEVMEDHAGTYGLKK

SEQ ID NO: 33 - protein, 20 aa, synthetic construct
  LIPID 1: note = palmitoylated
  LIPID 2: note = palmitoylated
  LIPID 19: note = palmitoylated
  LIPID 20: note = palmitoylated
  SITE 9: note = phosphorylation
  SITE 11: note = phosphorylation
KKPGSRSRTPSLPTPPTRKK

SEQ ID NO: 34 - protein, 20 aa, synthetic construct
  LIPID 1: note = palmitoylated
  LIPID 2: note = palmitoylated
  LIPID 19: note = palmitoylated
  LIPID 20: note = palmitoylated
  SITE 9: note = phosphorylation
  SITE 12: note = phosphorylation
KKGYSSPGSPGTPGSRSRKK

SEQ ID NO: 35 - protein, 22 aa, synthetic construct
  LIPID 1: note = palmitoylated
  LIPID 2: note = palmitoylated
  LIPID 21: note = palmitoylated
  LIPID 22: note = palmitoylated
  SITE 4: note = phosphorylation
  SITE 9: note = phosphorylation
KKGDTSPRHLSNVSSTGSIDKK

SEQ ID NO: 36 - protein, 21 aa, synthetic construct
  LIPID 1: note = palmitoylated
  LIPID 2: note = palmitoylated
  LIPID 20: note = palmitoylated
  LIPID 21: note = palmitoylated
  SITE 3: note = phosphorylation
  SITE 6: note = phosphorylation
  SITE 13: note = phosphorylation
  SITE 15: note = phosphorylation
KKPGSPGTPGSRSRTPSLPKK

SEQ ID NO: 37 - protein, 16 aa, synthetic construct
  LIPID 1: note = palmitoylated
  LIPID 2: note = palmitoylated
  LIPID 15: note = palmitoylated
  LIPID 16: note = palmitoylated
  SITE 3: note = phosphorylation
KKHLSNVSSTGSIDKK

SEQ ID NO: 38 - protein, 14 aa, synthetic construct
  LIPID 1: note = palmitoylated
  LIPID 2: note = palmitoylated
  LIPID 13: note = palmitoylated
  LIPID 14: note = palmitoylated
  SITE 8: note = phosphorylation
KKVSGDTSPRHLKK

SEQ ID NO: 39 - protein, 55 aa, synthetic construct; identical to SEQ ID NO: 13

SEQ ID NO: 40 - protein, 53 aa, synthetic construct
AKFVAAWTLKAAAGSQYIKANSKFIGITELGSFNNFTVSFWLRVPKVSASHLE

SEQ ID NO: 41 - protein, 75 aa, synthetic construct; identical to SEQ ID NO: 15

SEQ ID NO: 42 - protein, 53 aa, synthetic construct; identical to SEQ ID NO: 16

SEQ ID NO: 43 - protein, 53 aa, synthetic construct; identical to SEQ ID NO: 17

SEQ ID NO: 44 - protein, 57 aa, synthetic construct
  LIPID 56: note = palmitoylated
  LIPID 57: note = palmitoylated
  MOD_RES 57: note = C-terminal amide
AKFVAAWTLKAAAVVRQYIKANSKFIGITELVVRFNNFTVSFWLRVPKVSASHLEKK

SEQ ID NO: 45 - protein, 17 aa, synthetic construct
  REGION 1: note = Biotin-LC linker (Ahx)
  SITE 5: note = phosphorylation
  SITE 13: note = phosphorylation
  MOD_RES 17: note = C-terminal amide
GVYKSPVVSGDTSPRHL

SEQ ID NO: 46 - protein, 17 aa, synthetic construct
  REGION 1: note = Biotin-LC linker (Ahx)
  MOD_RES 17: note = C-terminal amide
GVYKSPVVSGDTSPRHL

SEQ ID NO: 47 - protein, 17 aa, synthetic construct
  REGION 1: note = Biotin-LC linker (Ahx)
  SITE 4: note = phosphorylation
  SITE 12: note = phosphorylation
  MOD_RES 17: note = C-terminal amide
VYKSPVVSGDTSPRHLS

SEQ ID NO: 48 - protein, 17 aa, synthetic construct
  REGION 1: note = Biotin-LC linker (Ahx)
  MOD_RES 17: note = C-terminal amide
VYKSPVVSGDTSPRHLS

(19) United States
(12) Patent Application Publication
(10) Pub. No.: US 2025/0127867 A1
(43) Pub. Date: Apr. 24, 2025

(54) METHOD OF TREATING PRECLINICIAL ALZHEIMER'S DISEASE

(71) Applicants: JANSSEN PHARMACEUTICALS, INC., Titusville, NJ (US); AC IMMUNE SA, Lausanne (CH)

(72) Inventors: Johannes Streffer, Lausanne (CH); Lennert Steukers, Pulle (BE); Lingjue LI, Newtown, PA (US); Iva KEZIC, Antwerp (BE); Ziad SAAD, Encinitas, CA (US); Tricia THORNTON-WELLS, Lincoln, MA (US); David HENLEY, Hagerstown, IN (US); Cathy BLEYS, Antwerp (BE)

(21) Appl. No.: 18/926,209

(22) Filed: Oct. 24, 2024

Related U.S. Application Data
(60) Provisional application No. 63/655,362, filed on Jun. 3, 2024.
(60) Provisional application No. 63/592,793, filed on Oct. 24, 2023.

(51) Int. Cl. A61K 39/00; A61P 25/28

(52) U.S. Cl. CPC A61K 39/0007; A61P 25/28; A61K 2039/54; A61K 2039/55555; A61K 2039/55561; A61K 2039/55572

(57) ABSTRACT

The application describes a phosphorylated tau targeted active immunotherapy to treat preclinical Alzheimer's Disease.

### CROSS-REFERENCE TO RELATED APPLICATION

This application claims priority to each of U.S. Provisional Patent Application No. 63/592,793, filed Oct. 24, 2023, and U.S. Provisional Patent Application No. 63/655,362, filed Jun. 3, 2024, the disclosure of each of the foregoing applications is incorporated herein by reference in its entirety.

### FIELD OF THE INVENTION

The present invention is in the field of medicine. The invention in particular relates to a method of treating preclinical Alzheimer's disease by using a liposome that contains a phosphorylated tau peptide.

### REFERENCE TO SEQUENCE LISTING SUBMITTED ELECTRONICALLY

This application contains a sequence listing, which is submitted electronically. The information contained in the electronic sequence listing (Sequence Listing 065768.11217.xml; size: 89 KB; and date of creation: Oct. 24, 2023) is incorporated herein by reference in its entirety.

### BACKGROUND OF THE INVENTION

Alzheimer's disease (AD) is a fatal neurodegenerative disease that is manifested by progressive deficits including memory loss followed by loss of independent function as well as neuropsychiatric symptoms such as apathy, depression, anxiety, agitation, and psychosis. There are limited disease modifying therapies for symptomatic AD. Lecanemab has obtained traditional approval; lecanemab and investigational therapies closest to market target amyloid, which is likely to slow disease progression by ˜30%, leaving a continued unmet need for treatments to further slow disease progression (Van Dyck et al., 2023, N Engl J Med. 388 (1): 9-21). A recent extensive literature review on prevalence across the AD continuum and a derived model estimating the number of persons with AD, suggests a total of 416 million persons worldwide are on the AD continuum (22% of all persons aged 50 and above), ranging between 327 and 525 million. Of those, the estimated number of persons with preclinical AD is 315 million, ranging between 258 and 376 million. This constituted 17% of all persons aged 50 and above, and 52% of those were women (Gustavsson et al., 2023, Alzheimers Dement. 19 (2): 658-670.). Age is the greatest risk factor for AD and with the increasing number of elderly in the global population, AD is a major medical and economic concern. In addition to factors like geographic origin, age, sex assigned at birth and comorbidity, racial and ethnic groups experience a significant burden of AD morbidity (Franzen et al., 2022, Alzheimers Dement. 18 (4): 810-823).

AD is a progressive neurocognitive disorder characterized by the presence of amyloid-containing plaques and hyperphosphorylated tau-containing neurofibrillary tangles in brain tissue on autopsy or, in living individuals, by in vivo neuroimaging and/or by analysis of cerebrospinal fluid (CSF) concentrations of amyloid and tau peptides. In AD and other tauopathies, such abnormally hyperphosphorylated tau accumulates as intraneuronal tangles of paired helical filaments (PHF), twisted ribbons and/or straight filaments (Iqbal 2005, Biochim Biophys Acta. 2005; 1739 (2-3): 198-210).

There remains an unmet need for safe and effective treatments for AD, in particular, a treatment to preclinical AD to prevent and/or delay progression of the preclinical AD into subsequent symptomatic stages of AD.

### SUMMARY OF THE INVENTION

The application describes a phosphorylated tau targeted active immunotherapy to treat preclinical Alzheimer's Disease.

Tau pathology is closely associated with neurodegeneration and cognitive decline. The sequential emergence of tau pathology across interconnected brain regions supports the hypothesis that tau pathology spreads in a “prion-like” fashion across connected neurons (Calafate et al., Cell Reports. 2015; 11 (8): 1176-1183; de Calignon et al., Cell Reports. 2015; 11 (8): 1176-1183; Franzmeier et al., Sci Adv. 2021; 7 (44): eabh1448; Meisl et al., Sci Adv. 2021; 7 (44): eabh1448). Tau aggregates can self-replicate, e.g., via local cell-to-cell transfer, growth, and/or multiplication. The replication-competent tau aggregates, also referred to as tau seeds or proteopathic seeds, can be transferred between brain regions. The spreading of tau seeds from one brain region to another has been hypothesized to be a key factor in the disease progression (Schoonhoven, et al., Brain, Volume 146, Issue 10, October 2023, Pages 4040-4054.). The identification of extracellular pathological phosphorylated tau (pTau) and its ability to spread across the brain provides opportunities to target pTau with antibodies.

Inventors of the application discovered that antibodies stimulated by a pTau vaccine block the seeding activity and the spreading of replication-competent tau aggregates between brain regions. Thus, administering the pTau vaccine to a subject having a preclinical AD, prior to substantial neuronal loss but close to symptom onset [optimal window for disease modification], slows cognitive decline, prevents or slows the transition from preclinical AD to mild cognitive impairment (MCI) in the subject.

Accordingly, a general aspect of the application relates to a method of treating preclinical Alzheimer's disease in a human subject in need thereof: The method comprises intramuscularly administering an effective amount of a liposome to the human subject, wherein the liposome comprises:
(i) a tau peptide having the amino acid sequence selected from the group consisting of SEQ ID NO: 1-12, wherein the tau peptide is presented on the surface of the liposome;
(ii) a helper T cell epitope comprising at least one amino acid sequence selected from the group consisting of: SEQ ID NOs: 23, 24, 25, and 26;
(iii) a lipidated CpG oligonucleotide, wherein the CpG oligonucleotide comprises one or more phosphorothioate internucleotide linkages, and wherein the CpG oligonucleotide is covalently linked to at least one cholesterol via a linker, and
(iv) monophosphoryl lipid A (MPLA).
   wherein the human subject has one or more of tau pathology and has no cognitive impairment. In some embodiments, the tau peptide has the amino acid sequence of SEQ ID NO: 2, 5, 9 or 12.

In certain embodiments, the liposome comprises:
(i) the tau peptide having the amino acid sequence of SEQ ID NO: 2, wherein the tau peptide is presented on the surface of the liposome;
(ii) the helper T cell epitope comprising at least one amino acid sequence selected from the group consisting of: SEQ ID NOs: 23, 24, 25, and 26;
(iii) the lipidated CpG oligonucleotide, wherein the CpG oligonucleotide comprises one or more phosphorothioate internucleotide linkages, and wherein the CpG oligonucleotide is covalently linked to at least one cholesterol via a linker; and
(iv) monophosphoryl lipid A (MPLA).

In certain embodiments, the liposome comprises:
a. the tau peptide has an amino acid sequence selected from the group consisting of SEQ ID NO: 28, 31, 35, and 38;
b. the helper T cell epitope comprises the amino acid sequences of SEQ ID NO: 23, SEQ ID NO: 24 and SEQ ID NO: 25, wherein the amino acid sequences are covalently linked together, optionally via one or more linkers; and
c. the lipidated CpG oligonucleotide has the nucleotide sequence selected from the group consisting of SEQ ID NO: 18 to SEQ ID NO: 22.

In some embodiments, the helper T cell epitope comprises an amino acid sequence selected from the group consisting of SEQ ID NOs: 39, 40, 41, 42, 43, 13, 14, 15, 16, 17, and 44.

In certain embodiments, the liposome comprises:
a. the tau peptide comprising the amino acid sequence of SEQ ID NO: 2;
b. the helper T cell epitope having an amino acid sequence selected from the group consisting of SEQ ID NOs: 39, 40, 41, 42, 43, 13, 14, 15, 16, 17, and 44.
c. the lipidated CpG oligonucleotide having the nucleotide sequence selected from the group consisting of SEQ ID NO: 18 to SEQ ID NO: 22, wherein the CpG oligonucleotide is covalently linked to at least one cholesterol via a linker; and
d. monophosphoryl lipid A (MPLA).

In certain embodiments, the liposome comprises:
a. the tau peptide has the amino acid sequence of SEQ ID NO: 28;
b. the helper T cell epitope having an amino acid sequence selected from the group consisting of SEQ ID NOs: 39, 40, 41, 42, and 43; and
c. the lipidated CpG oligonucleotide having a nucleotide sequence selected from the group consisting of SEQ ID NO: 18 to SEQ ID NO:22, wherein the CpG oligonucleotide is covalently linked to at least one cholesterol via a linker.

In certain embodiments, the liposome comprises:
a. the tau peptide comprises the amino acid sequence of SEQ ID NO: 28;
b. the helper T cell epitope has an amino acid sequence selected from the group consisting of SEQ ID NOs: 13, 14 or 15; and
c. the lipidated CpG oligonucleotide having the nucleotide sequence selected from the group consisting of SEQ ID NO: 18.

In some embodiments, the liposome further comprises one or more lipids selected from the group consisting of 1,2-dimyristoyl-sn-glycero-3-phosphocholine (DMPC), 1,2-dimyristoyl-sn-glycero-3-phosphoryl-3′-rac-glycerol (DMPG), and cholesterol.

In some embodiments, the human subject has a tau pathology as measured by an elevated plasma level of p-tau217, p-tau181, and/or p-tau231, and/or by a tau positron emission tomography (PET) scan at the initial administration of the effective amount of the liposome.

In some embodiments, the human subject (i) has an elevated brain tau pathology defined as Braak III region of interest standardized uptake value ratio (Braak 3 ROI SUVR)>1.1 on a screening tau PET scan, and (ii) has no diagnosis of Alzheimer's Dementia or non-Alzheimer's Dementia or Mild Cognitive Impairment, at the initial administration of the effective amount of the liposome.

In some embodiments, the human subject has no diagnosis of Alzheimer's Dementia or non-Alzheimer's Dementia or Mild Cognitive Impairment, and has at least one of (i) an elevated brain tau pathology defined as Braak III region of interest standardized uptake value ratio (Braak 3 ROI SUVR)>1.1 on a screening tau PET scan, (ii) a Clinical Dementia Rating (CDR) global score of 0, and/or (iii) a Mini-Mental State Examination (MMSE) score of 25 or more, at the initial administration of the effective amount of the liposome.

In certain embodiments, the human subject has (i) an elevated brain tau pathology defined as Braak III region of interest standardized uptake value ratio (Braak 3 ROI SUVR)>1.1 on a screening tau PET scan, (ii) a CDR global score of 0, and (iii) a MMSE score of 25 or more, at the initial administration of the effective amount of the liposome.

In certain embodiments, the human subject has (i) an elevated brain tau pathology defined as Braak III region of interest standardized uptake value ratio (Braak 3 ROI SUVR)>1.1 on a screening tau PET scan, (ii) a CDR global score of 0, and (iii) a MMSE score of 27 or more, at the initial administration of the effective amount of the liposome.

In some embodiments, the human subject has (iii) a Clinical Dementia Rating (CDR) global score of 0, and/or (iv) a Mini-Mental State Examination (MMSE) score of 25 or more, at the initial administration of the effective amount of the liposome.

In some embodiments, at the time of the initial treatment, the human subject is 75 years of age or younger, or 50 or 55 years of age or older, particularly the human subject is at least 50, 51, 52, 53, 54, 55, 56, 57, 58, 59 or 60 years of age, more particularly 55-75 years of age at the time of the initial treatment. In some embodiments, at the time of the initial treatment, the human subject has an MMSE score of 27 or more. In some embodiments, at the time of the initial treatment, one or more Alzheimer's disease-related biomarkers is/are present in the human subject. In some embodiments, at the time of the initial treatment, the human subject has, for example, an elevated plasma phosphorylated tau (p-tau) level, particularly an elevated p-tau217, an elevated p-tau181, and/or an elevated p-tau231. In some embodiments, the human subject having preclinical AD has a brain amyloid pathology. An elevated plasma p-tau217 level is also predictive of amyloid pathology.

According to embodiments of the application, the effective amount of the liposome comprises 300 μg to 1800 μg per dose of the tau peptide, and the liposome is administered to the human subject subcutaneously or intramuscularly. In some embodiments, the effective amount of the liposome comprises 300, 900 or 1800 μg per dose of the Tau peptide, and the liposome is administered to the human subject intramuscularly.

In some embodiments, an effective amount of a liposome is intramuscularly administered to the human subject once every 6 months for a period of at least 1 year. In some embodiments, after the administration on Day 1, the effective amount of the liposome is administered to the human subject repeatedly, e.g., at week 8, weeks 24, week 50, and every 26 weeks after week 50.

In some embodiments, the method further comprises: (a) intramuscularly administering the effective amount of the liposome to the human subject 24 weeks before initiating the once every 6 months administrations, and (b) intramuscularly administering the effective amount of the liposome to the human subject 16 weeks before initiating the once every 6 months administrations.

In some embodiments, the effective amount of the liposome is administered to the human subject once every 6 months for a period of at least 1.5 years, at least 2 years, at least 2.5 years, at least 3 years, at least 3.5 years or at least 4 years.

In some embodiments, the method comprises: (a) intramuscularly administering the effective amount of a liposome to the human subject at week 0 of the treatment; (b) intramuscularly administering the effective amount of the liposome to the human subject at week 8 of the treatment; and (c) intramuscularly administering the effective amount of the liposome to the human subject once every 6 months for a period of at least 1 year, wherein the period starts at week 24 of the treatment.

In some embodiments, the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

In another general aspect, the application provides a method of treating preclinical Alzheimer's disease in a human subject in need thereof, comprising intramuscularly administering an effective amount of a liposome to the human subject once every 6 months for a period of at least 1 year, wherein the liposome comprises:
(i) a tau peptide having the amino acid sequence of SEQ ID NO: 2, wherein the tau peptide is presented on the surface of the liposome;
(ii) a helper T cell epitope comprising at least one amino acid sequence selected from the group consisting of: SEQ ID NOs: 23, 24, 25, and 26;
(iii) a lipidated CpG oligonucleotide, wherein the CpG oligonucleotide comprises one or more phosphorothioate internucleotide linkages, and wherein the CpG oligonucleotide is covalently linked to at least one cholesterol via a linker; and
(iv) monophosphoryl lipid A (MPLA).

In another general aspect, the application provides a method of treating preclinical Alzheimer's disease in a human subject in need thereof, comprising intramuscularly administering an effective amount of a liposome to the human subject once every 6 months for a period of at least 1 year, wherein the liposome comprises:
(v) a tau peptide having the amino acid sequence of SEQ ID NO: 2, wherein the tau peptide is presented on the surface of the liposome;
(vi) a helper T cell epitope comprising at least one amino acid sequence selected from the group consisting of: SEQ ID NOs: 23, 24, 25, and 26;
(vii) a lipidated CpG oligonucleotide, wherein the CpG oligonucleotide comprises one or more phosphorothioate internucleotide linkages, and wherein the CpG oligonucleotide is covalently linked to at least one cholesterol via a linker; and
(viii) monophosphoryl lipid A (MPLA);
wherein the human subject has a tau pathology and no cognitive impairment.

In another general aspect, the application provides a method of treating preclinical Alzheimer's disease in a human subject, comprising:
(a) intramuscularly administering an effective amount of a liposome to the human subject at week 0 of the treatment;
(b) intramuscularly administering the effective amount of the liposome to the human subject at week 8 of the treatment; and
(c) intramuscularly administering the effective amount of the liposome to the human subject once every 6 months for a period of at least 1 year, wherein the period starts at week 24 of the treatment,
wherein the liposome comprises:
(i) a tau peptide having the amino acid sequence of SEQ ID NO: 28, wherein the tau peptide is presented on the surface of the liposome;
(ii) a helper T cell epitope having an amino acid sequence selected from the group consisting of SEQ ID NOs: 39, 40, 41, 42, and 43;
(iii) a lipidated CpG oligonucleotide having a nucleotide sequence selected from the group consisting of SEQ ID NO:18 to SEQ ID NO:22, wherein the CpG oligonucleotide is covalently linked to at least one cholesterol via a linker; and
(iv) monophosphoryl lipid A (MPLA),
wherein the human subject has a tau pathology and no cognitive impairment.

In another general aspect, the application provides a method of treating preclinical Alzheimer's disease in a human subject, comprising:
(a) intramuscularly administering an effective amount of a liposome to the human subject at week 0 of the treatment;
(b) intramuscularly administering the effective amount of the liposome to the human subject at week 8 of the treatment; and
(c) intramuscularly administering the effective amount of the liposome to the human subject once every 6 months for a period of at least 1 year, wherein the period starts at week 24 of the treatment,
wherein the liposome comprises:
(i) a tau peptide comprising the amino acid sequence of SEQ ID NO: 28, wherein the tau peptide is presented on the surface of the liposome;
(ii) a helper T cell epitope having an amino acid sequence selected from the group consisting of SEQ ID NOs: 13, 14 or 15;
(iii) a lipidated CpG oligonucleotide having the nucleotide sequence selected from the group consisting of SEQ ID NO: 18, wherein the CpG oligonucleotide is covalently linked to at least one cholesterol via a linker, and
(iv) monophosphoryl lipid A (MPLA).
wherein the human subject is 50-75 years of age and has a tau pathology as measured by an elevated plasma level of p-tau217 and a tau PET scan at week 0 and has no cognitive impairment.

In certain embodiments, a method of the application further comprises detecting one or more biomarkers related to Alzheimer's disease before, during, and/or after the administering step.

Preferably, a treatment according to a method of the application results in prevention and/or delay of progression of the preclinical AD into subsequent symptomatic stages of AD. In particular, a method according to an embodiment of the application prevents and/or delays the progression of the preclinical AD as assessed by one or more of (1) Preclinical Alzheimer's Cognitive Composite-5 (PACC-5) total score; (2) tau PET scans; and (3) other clinical outcomes, such as, but not limited to, Mild Behavioral Impairment Checklist (MBI-C), Activities of Daily Living Prevention Instrument (ADCS-ADL-PD), Clinical Dementia Rating (CDR)-Global Score (GS) and CDR-sum of boxes scores (CDR-SB).

In certain embodiments, a method of the application mitigates or lessens neuropathology in a human subject having preclinical AD, particularly, the method blocks the seeding activity of PHF or enriched PHF (ePHF) and prevents the formation and/or accumulation of Tau aggregates.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows the capacity of antibodies generated after three immunizations with ACI-35.030 active immunotherapy or ACI-35E.030 placebo to reduce the formation of de-novo rat Tau aggregates in primary rat cortical neurons. Data is expressed as percent reduction of rat Tau aggregates present in primary rat cortical neurons after incubation with ePHF in the presence of several dilutions of post-dose (Day 99) as compared to pre-dose (Day-14) monkey serum.

FIG. 2 shows the capacity of antibodies generated after two and four immunizations with ACI-35.030 active immunotherapy to reduce the formation of de-novo rat Tau aggregates in primary rat cortical neurons. Data is expressed as percent reduction of rat Tau aggregates present in primary rat cortical neurons after incubation with ePHF in the presence of several dilutions of post-dose (Day 50 or 190, respectively) as compared to pre-dose (Day-63) monkey serum.

FIG. 3 shows that serum from ACI-35.030 immunized rhesus monkeys can block AD-Tau seed uptake as seen by significant reduction of aggregation levels of endogenous rodent Tau. Graph represents estimated mean % of remaining aggregation of Tau normalized to the levels of Tau aggregation when only AD-Tau seeds are added. Measured Tau aggregation levels are normalized to aSyn to control for possible differences in number of cells per well due to plate variation. Estimated mean Tau % remaining seeding values and 95% confidence intervals are shown by day and dilution combination, and p-values of the differences (with Sidak correction) based on fitted mixed-effects model. (*p-value≤0.05, **p-value≤0.01 and ***p-value≤0.001). In each group of three bars, the left bar represents d-14 results, middle bar d50 results and right bar d190 results.

FIG. 4 illustrates a schematic overview of the study design for the Phase 2b study.

### DETAILED DESCRIPTION OF THE INVENTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications, and publications cited herein are incorporated by reference as if set forth fully herein.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The application provides a method of treating preclinical Alzheimer's disease in a human subject in need thereof, comprising administering to the subject an effective amount of liposomes comprising a Tau peptide presented on the surface of the liposome and a toll-like receptor 4 agonist.

As used herein, “preclinical Alzheimer's disease” or “preclinical AD” refers to a preclinical stage of Alzheimer's disease which is part of the 2018 National Institute on Aging-Alzheimer's Association (NIA-AA) Research Framework NIA-AA Research Framework definition for AD, and this preclinical stage of AD is globally defined by the absence of clinical symptoms of AD (absence of cognitive impairment and absence of functional impairment) while the neuropathology related to AD is already present in the brain based on the AT (N) classification that relates respectively to beta-amyloid deposition (A), pathological Tau (T) and neurodegeneration (N). In the preclinical stage of AD, the beta-amyloid deposition is always present (e.g., aggregated Abeta or associated pathologic state CSF Ab42, or Ab42/Ab40 ratio, Amyloid PET) while, depending on the stage in the continuum of Alzheimer's pathology, the presence of pathological Tau (e.g., aggregated tau (neurofibrillary tangles) or associated pathological state CSF phosphorylated tau) and the presence of neurodegeneration (e.g., neurodegeneration or neuronal injury anatomic MRI FDG PET CSF total tau) may be present or absent.

As used herein, “treating preclinical Alzheimer's disease” or a “treatment of preclinical Alzheimer's disease” refers to a treatment that prevents and/or delays progression of the preclinical AD into subsequent symptomatic stages of AD.

In some embodiments, a treatment of preclinical AD prevents and/or delays the onset of clinical symptoms of AD. In some embodiments, a treatment of preclinical AD prevents and/or delays the onset of cognitive impairment related to AD. As used herein, “cognitive impairment” or “cognitive decline” refers to the concern of or difficulty with a person's thinking, memory, concentration, and/or other brain functions beyond what is typically expected due to aging. While by definition, a human subject having preclinical AD does not yet have cognitive impairment or cognitive decline, the human subject may have a decline in his/her brain function, such as some decline in his/her semantic memory. A method according to an embodiment of the application can prevent or delay the decline or reduce the rate of the decline of the human subject's cognitive or brain function. The human subject's cognitive or brain function can be evaluated using methods known in the art in view of the present disclosure. In certain embodiment, a method of the application results in prevention and/or delay of the onset of cognitive impairment related to AD as measured by Preclinical Alzheimer's Cognitive Composite-5 (PACC-5) total score.

In some embodiments, a treatment of preclinical AD prevents and/or delays the onset of functional impairment related to AD. Functional impairment can be, for example, the decline in performance of Activities of Daily Living (ADL). Functional activities can be assessed using methods known in the art in view of the present disclosure. In certain embodiment, a method of the application results in prevention and/or delay of the onset of functional impairment related to AD as measured by, for example, Mild Behavioral Impairment Checklist (MBI-C), Activities of Daily Living Prevention Instrument (ADCS-ADL-PI), CDR-GS and CDR-SB.

In some embodiments, a treatment of preclinical AD mitigates or lessens neuropathology related to AD in a human subject having the preclinical AD. In certain embodiment, a method of the application blocks the seeding activity of PHF or enriched PHF (ePHIF) and prevents the formation and/or accumulation of pathological Tau (e.g., aggregated tau or neurofibrillary tangles). The pathological Tau can be measured using methods known in the art in view of the present disclosure, such as tau PET scans. The treatment can also reduce phosphorylated tau associated with the pathological Tau in the CSF or plasma.

In one general aspect, the application relates to a method of treating preclinical AD in a human subject in need thereof: The method comprises administering to the subject an effective amount of liposomes comprising an amount of Tau phosphopeptide that is sufficient to induce a sustained immune response against phosphorylated Tau, such as an increased level of anti-phosphorylated Tau antibodies, without inducing a serious adverse event related to the liposomes, such as encephalitis, and the sustained immune response against phosphorylated Tau serves to prevent and/or to delay the progression of preclinical AD into the symptomatic stages of AD. The measure of the delay in the appearance of, or, of the prevention of the progression from preclinical AD into symptomatic stages of AD can be evaluated by one or more assessment tools, such as one or more of (1) Preclinical Alzheimer's Cognitive Composite-5 (PACC-5) total global score; (2) tau PTE scans; and (3) other clinical outcomes, such as, but not limited to, Mild Behavioral Impairment Checklist (MBI-C), Activities of Daily Living Prevention Instrument (ADCS-ADL-PI), Clinical Dementia Rating (CDR)-Global Score (GS) and CDR-sum of boxes scores (SB).

As used herein, the term “tau” or “tau protein”, also known as microtubule-associated protein Tau, MAPT, neurofibrillary tangle protein, paired helical filament (PHF)-Tau, enriched paired helical filament (ePHF)-Tau, MAPTL, MTBTI, refers to an abundant central and peripheral nervous system protein having multiple isoforms. In the human central nervous system (CNS), six major Tau isoforms ranging in size from 352 to 441 amino acids in length exist due to alternative splicing (Hanger et al., Trends Mol Med. 15:112-9, 2009). Examples of Tau include, but are not limited to, Tau isoforms in the CNS, such as the 441-amino acid longest Tau isoform (4R2N), also named microtubule-associated protein tau isoform 2, that has four repeats and two inserts, such as the human Tau isoform 2 having the amino acid sequence represented in GenBank Accession No. NP_005901.2. Other examples of Tau include the 352-amino acid long shortest (fetal) isoform (3R0N), also named microtubule-associated protein tau isoform 4, that has three repeats and no inserts, such as the human Tau isoform 4 having the amino acid sequence represented in GenBank Accession No. NP_058525.1. Examples of Tau also include the “big Tau” isoform expressed in peripheral nerves that contains 300 additional residues (exon 4a). Friedhoff et al., Biochimica et Biophysica Acta 1502 (2000) 122-132. Examples of Tau include a human big Tau that is a 758 amino acid-long protein encoded by an mRNA transcript 6762 nucleotides long (NM_016835.4), or isoforms thereof: The amino acid sequence of the exemplified human big Tau is represented in GenBank Accession No. NP 058519.3. As used herein, the term “Tau” includes homologs of Tau from species other than human, such as Macaca Fascicularis (cynomolgus monkey), rhesus monkeys or Pan troglodytes (chimpanzee). As used herein, the term “Tau” includes proteins comprising mutations, e.g., point mutations, fragments, insertions, deletions and splice variants of full-length wild type Tau. The term “Tau” also encompasses post-translational modifications of the Tau amino acid sequence. Post-translational modifications include, but are not limited to, phosphorylation.

As used herein, the term “tau pathology” refers to a condition that occurs when tau protein becomes abnormally hyperphosphorylated and aggregates in the brain, which can lead to neurodegenerative diseases. Tau pathology is closely associated with neurodegeneration and cognitive decline. The sequential emergence of tau pathology across interconnected brain regions supports the hypothesis that tau pathology spreads in a “prion-like” fashion across connected neurons (Calafate et al., Cell Reports. 2015; 11 (8): 1176-1183; de Calignon et al., Cell Reports. 2015; 11 (8): 1176-1183; Franzmeier et al., Sci Adv. 2021; 7 (44): eabh1448; Meisl et al., Sci Adv. 2021; 7 (44): eabh1448).

As used herein, the term “peptide” or “polypeptide” refers to a polymer composed of amino acid residues, related naturally occurring structural variants, and synthetic non-naturally occurring analogs thereof linked via peptide bonds. The term refers to a peptide of any size, structure, or function. Typically, a peptide is at least three amino acids long. A peptide can be naturally occurring, recombinant, or synthetic, or any combination thereof: Synthetic peptides can be synthesized, for example, using an automated polypeptide synthesizer. Examples of Tau peptides include any peptide of Tau protein of about 5 to about 30 amino acids in length, preferably of about 10 to about 25 amino acids in length, more preferably of about 16 to about 21 amino acids in length. In the present disclosure, peptides are listed from N to C terminus using the standard three or one letter amino acid abbreviation, wherein phosphoresidues are indicated with “p”. Examples of Tau peptides useful in the invention include, but are not limited to, Tau peptides comprising the amino acid sequence of any of SEQ ID NOs: 1-12, or Tau peptides having an amino acid sequence that is at least 75%, 80%, 85%, 90% or 95% identical to the amino acid sequence of any of SEQ ID NOs: 1-12.

As used herein, the term “phosphopeptide” or “phospho-epitope” refers to a peptide that is phosphorylated at one or more amino acid residues. Examples of Tau phosphopeptides include any Tau peptide comprising one or more phosphorylated amino acid residues.

The Tau peptides of the present invention can be synthesized by solid phase peptide synthesis or by recombinant expression systems. Automatic peptide synthesizers are commercially available from numerous suppliers, such as Applied Biosystems (Foster City, Calif.). Recombinant expression systems can include bacteria, such as E. coli, yeast, insect cells, or mammalian cells. Procedures for recombinant expression are described by Sambrook et al., Molecular Cloning: A Laboratory Manual (C.S.H.P. Press, NY 2d ed., 1989).

As used herein, the term “liposome” refers generally to a lipid vesicle that is made of materials having high lipid content, e.g., phospholipids, cholesterol. The lipids of these vesicles are generally organized in the form of lipid bilayers. The lipid bilayers generally encapsulate a volume which is either interspersed between multiple onion-like shells of lipid bilayers, forming multilamellar lipid vesicles (ML Vs) or contained within an amorphous central cavity. Lipid vesicles having an amorphous central cavity are unilamellar lipid vesicles, i.e., those with a single peripheral bilayer surrounding the cavity. Large unilamellar vesicles (LUVs) generally have a diameter of 100 nm to few micrometers, such as 100-200 nm or larger, while small unilamellar lipid vesicles (SUV) generally have a diameter of less than 100 nm, such as 20-100 nm, typically 15-30 nm.

According to embodiments of the application, a Tau peptide is presented on the surface of the liposome. A Tau peptide, preferably a Tau phosphopeptide, can be presented on the surface of the liposome using methods known in the art in view of the present disclosure. See, for example, the relevant disclosure in U.S. Pat. Nos. 8,647,631 and 9,687,447, and International Patent Application No. PCT/US18/57286, the content of which is incorporated herein by reference. According to particular embodiments, the one or more Tau peptides, including phosphopeptides, further comprise one or more modifications, such as palmitoylation or dodecyl modification to allow the Tau peptides to be presented on the surface of the liposome. Additional amino acid residues, such as Lys, Cys, or sometimes Ser or Thr, can be added to the Tau peptide to facilitate the modification. It was reported that the position of lipid anchors induces different conformations of the peptide sequence (Hickman et al., J. Biol. Chem. vol. 286, No. 16, pp. 13966-13976, Apr. 22, 2011). While not wishing to be bound by theory, it is believed that adding hydrophobic moieties at both termini may increase the pathological beta-sheet conformation of the Tau peptide. Thus, the one or more Tau peptides further comprise hydrophobic moieties at both termini. The modified Tau peptide can have the C-terminus amidated. Preferably, a Tau peptide presented on the surface of the liposome consists of the amino acid sequence of one of SEQ ID NO:27 to SEQ ID NO:29 and SEQ ID NO:31 to SEQ ID NO: 38.

Examples of tau liposomes useful for the present invention include, but are not limited to, tau liposomes described in U.S. Pat. Nos. 8,647,631 and 9,687,447, and International Patent Application No. PCT/US18/57286, the disclose of each is herein incorporated by reference in its entirety.

Preferably, the liposome further comprises one or more lipids selected from the group consisting of 1,2-dimyristoyl-sn-glycero-3-phosphocholine (DMPC), 1,2-dimyristoyl-sn-glycero-3-phosphoryl-3′-rac-glycerol (DMPG), and cholesterol.

As used herein, the terms “induce” and “stimulate” and variations thereof refer to any measurable increase in cellular activity. Induction of an immune response can include, for example, activation, proliferation, or maturation of a population of immune cells, increasing the production of a cytokine, and/or another indicator of increased immune function. In certain embodiments, induction of an immune response can include increasing the proliferation of B cells, producing antigen-specific antibodies, increasing the proliferation of antigen-specific T cells, improving dendritic cell antigen presentation and/or an increasing expression of certain cytokines, chemokines and co-stimulatory markers.

As used herein a “sustained immune response” or a “sustainable immune response” refers to an immune response that lasts at least six weeks after the initial administration of an effective amount of a liposome. According to embodiments of the application, a “sustained immune response” is a sustained antibody response that lasts at least six weeks, at least 12 weeks, at least 24 weeks, at least 36 weeks, at least 48 weeks, at least 60 weeks, at least 72 weeks or longer, and the antibody response can be characterized by the presence of anti-phosphorylated Tau IgG, anti-phosphorylated Tau IgM, or anti-ePHF IgG. Anti-phosphorylated Tau IgG, anti-phosphorylated Tau IgM and anti-ePHE IgG can be detected and measured by any method known to one of skill in the art, including those described herein.

As used herein an “antibody response that lasts” refers to an antibody response that is maintained at a level equal to or higher than a defined threshold level during a specified period of time after the initial administration of an effective amount of a liposome, and the defined threshold level is higher than a baseline level measured before the initial administration of the effective amount of the liposome. In some embodiments, the baseline level is determined based on the average measured level of antibody titers before the initial administration, preferably two measurements are performed. In one embodiment, the antibody response comprises a specific IgG antibody response directed against the pTau, and the defined threshold level is at least 1.5 or more times of the baseline level, such as at least 1.5, 1.6, 1.7, 1.8, 1.9, 2.0 or more times of the baseline level. In another embodiment, the antibody response comprises an IgG immune response against ePHF, and the defined threshold level is at least 2.0 or more times of the baseline level, such as at least 2.0, 2.1, 2.2, 2.3, 2.4, 2.5 or more times of the baseline level.

As used herein, the term “anti-phosphorylated Tau antibody” refers to an antibody that binds to Tau that has been phosphorylated on an amino acid residue at one or more locations of the amino acid sequence of Tau. The phosphorylated amino acid residues can be, e.g., serine (Ser), threonine (Thr) or tyrosine (Tyr). The site on phosphorylated Tau to which the anti-phosphorylated Tau antibody binds is preferably a site that is specifically phosphorylated in neurodegenerative diseases such as Alzheimer's Disease. Examples of sites of phosphorylated Tau to which the anti-phosphorylated Tau antibody binds include, for example, Tyr18, Thr181, Ser199, Ser202, Thr205, Thr212, Thr217, Ser214, Ser396, Ser404, Ser409, Ser422, Thr427. As used throughout the present application, the amino acid positions are given in reference to the sequence of human microtubule-associated protein tau isoform 2 having the amino acid sequence represented in GenBank Accession No. NP_005901.2.

The ability to induce anti-phosphorylated Tau antibodies upon administration can be determined by testing a biological sample (e.g., blood, plasma, serum, PBMCs, urine, saliva, feces, CSF or lymph fluid) from the subject for the presence of antibodies, e.g., IgG or IgM antibodies, directed to the immunogenic Tau peptide(s) administered in the pharmaceutical composition (see for example Harlow, 1989, Antibodies, Cold Spring Harbor Press). For example, titers of antibodies produced in response to administration of a composition providing an immunogen can be measured by enzyme-linked immunosorbent assay (ELISA), other ELISA-based assays (e.g., MSD-Meso Scale Discovery), dot blots, SDS-PAGE gels, ELISPOT or Antibody-Dependent Cellular Phagocytosis (ADCP) Assay.

As used herein, the term “adverse event” (AB) refers to any untoward medical occurrence in a patient administered a pharmaceutical product and which does not necessarily have a causal relationship with the treatment. According to embodiments of the invention, AEs are rated on a 3-point scale of increasing severity using the following definitions: mild (grade 1), referring to an AE that is easily tolerated by the subject, which causes minimal discomfort and does not interfere with everyday activities; moderate (grade 2), referring to an AE that is sufficiently discomforting to interfere with normal everyday activities and intervention may be needed; severe (grade 3), referring to an AE that prevents normal everyday activities, and treatment or other intervention is usually needed. A serious AE (SAE) can be any AE occurring at any dose that results in any of the following outcomes: death, where death is an outcome, not an event; life-threatening, referring to an event in which the patient is at risk of death at the time of the event; it does not refer to an event which could hypothetically have caused death had it been more severe; in patient hospitalization, i.e., an unplanned, overnight hospitalization, or prolongation of an existing hospitalization; persistent or significant incapacity or substantial disruption of the ability to conduct normal life functions; congenital anomaly/birth defect; important medical event (as deemed by the investigator) that may jeopardize the patients or may require medical or surgical intervention to prevent one of the other outcomes listed above (e.g., intensive treatment in an emergency room or at home for allergic bronchospasm or blood dyscrasias or convulsions that do not result in hospitalization).

As used herein, the term “effective amount” refers to an amount of an active ingredient or component that elicits the desired biological or medicinal response in a subject. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the mode of administration, route of administration, target site, physiological state of the patient, other medications administered and the severity of disease, and should be decided according to the judgment of the practitioner and each patient's circumstances. For example, the effective amount of tau phosphopeptide also depends on whether adjuvant is also administered, with higher dosages being required in the absence of adjuvant. Effective doses can be extrapolated from dose-response curves derived from in vitro or animal model test systems.

In particular embodiments, an effective amount of liposomes comprises a Tau phosphopeptide at an amount of about 25 nmoles to about 750 nmoles per dose, such as about 29.7 nmoles to about 742.5 nmoles per dose, preferably about 90 nmoles to about 715 nmoles per dose, such as about 89.1 nmoles to about 712.8 nmoles per dose, or about 90 nmoles to about 535 nmoles per dose, such as about 89.1 nmoles to about 534.6 nmoles per dose, or about 90 nmoles to about 275 nmoles per dose, such as about 89.1 nmoles to about 267.3 nmoles per dose. The amount of Tau phosphopeptide administered can also be expressed by weight. For example, 29.7 nmoles per dose corresponds to 100 μg per dose of a tetrapalmitoylated Tau phosphopeptide consisting of the amino acid sequence of SEQ ID NO: 28, 742.5 nmoles per dose corresponds to 2500 μg per dose of a tetrapalmitoylated Tau phosphopeptide consisting of the amino acid sequence of SEQ ID NO: 28, 89.1 nmoles per dose corresponds to 300 μg per dose of a tetrapalmitoylated Tau phosphopeptide consisting of the amino acid sequence of SEQ ID NO: 28, 712.8 nmoles per dose corresponds to 2400 μg per dose of a tetrapalmitoylated Tau phosphopeptide consisting of the amino acid sequence of SEQ ID NO: 28, and 534.6 nmoles per dose corresponds to 1800 μg per dose of a tetrapalmitoylated Tau phosphopeptide consisting of the amino acid sequence of SEQ ID NO: 28. The tetrapalmitoylated Tau phosphopeptide has four lipidic chains that allow the presentation of the Tau phosphopeptide on the surface of the liposomes. The doses of 300, 900, 1800 μg of tetrapalmitoylated Tau phosphopeptide consisting of the amino acid sequence of SEQ ID NO: 28 correspond to 169, 508, 1016 μg, respectively of the corresponding “naked” peptide without any of the lipidic chains.

According to embodiments of the application, an effective amount of liposomes comprises a Tau phosphopeptide at an amount of about 25 nmoles to about 750 nmoles per dose, such as about 25 nmoles, about 30 nmoles, about 35 nmoles, about 40 nmoles, about 45 nmoles, about 50 nmoles, about 55 nmoles, about 60 nmoles, about 65 nmoles, about 70 nmoles, about 75 nmoles, about 80 nmoles, about 85 nmoles, about 90 nmoles, about 95 nmoles, about 100 nmoles, about 125 nmoles, about 150 nmoles, about 175 nmoles, about 200 nmoles, about 225 nmoles, about 250 nmoles, about 275 nmoles, about 300 nmoles, about 325 nmoles, about 350 nmoles, about 375 nmoles, about 400 nmoles, about 425 nmoles, about 450 nmoles, about 475 nmoles, about 500 nmoles, about 525 nmoles, about 550 nmoles, about 575 nmoles, about 600 nmoles, about 625 nmoles, about 650 nmoles, about 675 nmoles, about 700 nmoles, about 725 nmoles, about 750 nmoles per dose of a Tau phosphopeptide comprising the amino acid sequence of one of SEQ ID NOs: 1-3 or 5-12. Preferably, the Tau phosphopeptide consists of the amino acid sequence of one of SEQ ID NO:27 to SEQ ID NO:29 and SEQ ID NO:31 to SEQ ID NO: 38. More preferably, the Tau phosphopeptide consists of the amino acid sequence of SEQ ID NO: 28.

According to embodiments of the application, an effective amount of liposomes comprises a tetrapalmitoylated Tau phosphopeptide at an amount of 100 μg to 2500 μg, 300 μg to 2400 μg, 300 μg to 1800 μg, or 300 μg to 900 μg per dose, such as 100 μg, 150 μg, 200 μg, 250 μg, 300 μg, 400 μg, 500 μg, 600 μg, 700 μg, 800 μg, 900 μg, 1000 μg, 1100 μg, 1200 μg, 1300 μg, 1400 μg, 1500 μg, 1600 μg, 1700 μg, 1800 μg, 1900 μg, 2000 μg, 2100 μg, 2200 μg, 2300 μg, 2400 μg, or 2500 μg per dose.

As used herein, the term “toll-like receptor” or “TLR” refers to a class of pattern recognition receptor (PRR) proteins that play a key role in the innate immune response. TLRs recognize pathogen-associated molecular patterns (PAMPs) from microbial pathogens, such as bacteria, fungi, parasites and viruses, which can be distinguished from host molecules. TLRs are membrane-spanning proteins that typically function as dimers and are expressed by cells involved in the innate immune response, including antigen-presenting dendritic cells and phagocytic macrophages. There are at least ten human TLR family members, TLR1 to TLR10, and at least twelve murine TLR family members, TLR1 to TLR9 and TLR11 to TLR13, and they differ in the types of antigens they recognize. For example, TLR4 recognizes lipopolysaccharides (LPS), a component present in many Gram-negative bacteria, as well as viral proteins, polysaccharide, and endogenous proteins such as low-density lipoprotein, beta-defensins and heat shock protein; and TLR9 is a nucleotide-sensing TLR which is activated by unmethylated cytosine-phosphate-guanine (CpG) single-stranded or double-stranded dinucleotides, which are abundant in prokaryotic genomes but rare in vertebrate genomes. Activation of TLRs leads to a series of signaling events resulting in the production of type I interferons (IFNs), inflammatory cytokines, and chemokines, and the induction of immune responses. Eventually, this inflammation also activates the adaptive immune system, which then results in the clearance of the invading pathogens and the infected cells.

As used herein, the term “toll-like receptor 4 agonist” refers to any compound that acts as an agonist of TLR4. Any suitable toll-like receptor 4 agonist known to those skilled in the art in view of the present disclosure can be used in the invention. Examples of toll-like receptor 4 ligand useful for the invention include TLR4 agonist, including, but not limited to, monophosphoryl lipid A (MPLA). As used herein, the term “monophosphoryl lipid A” or “MPLA” refers to a modified form of lipid A, which is the biologically active part of Gram-negative bacterial lipopolysaccharide (LPS) endotoxin. MPLA is less toxic than LPS while maintaining the immunostimulatory activity. As a vaccine adjuvant, MPLA stimulates both cellular and humoral responses to the vaccine antigen. Examples of MPLA include, but are not limited to, 3-O-desacyl-4-monophosphoryl lipid A, Monophosphoryl Hexa-acyl Lipid A, 3-Deacyl (Synthetic) (also referred to as 3D-(6-acyl) PHAD®), monophosphoryl 3-deacyl lipid A, and structurally related variants thereof: MPLA useful for the invention can be obtained using methods known in the art, or from a commercial source, such as 3D-(6-acyl) PHAD®, PHAD®, PHAD®-504, 3D-PHAD® from Avanti Polar Lipids (Alabaster, Alabama, USA) or MPLTM from various commercial sources. According to particular embodiments, the toll-like receptor 4 agonist is MPLA.

According to particular embodiments, the liposome comprising a Tau phosphopeptide and a toll-like receptor 4 agonist also comprises a helper T-cell epitope that is capable of binding most or all HLA DR (Human Leukocyte Antigen-antigen D Related) molecules. The helper T-cell epitope is then able to activate CD4+ T-cells and provides essential maturation and survival signals to the Tau-specific B-cells. The Tau liposomes can be used to generate high-quality antibodies against the pTau antigen in homologous or heterologous immunization schemes, with the liposome used in the prime and/or in the boost.

According to other embodiments of the application, an effective amount of liposomes further comprises a toll-like receptor 4 agonist at an amount of 30 μg to 900 μg, preferably 100 μg to 585 μg, per dose. For example, the effective amount of liposomes can comprise a toll-like receptor 4 agonist at an amount of 30 μg, 50 μg, 100 μg, 150 μg, 200 μg, 250 μg, 300 μg, 330 μg. 360 μg, 390 μg, 420 μg, 450 μg. 480 μg. 500 μg, 520 μg, 540 μg, 560 μg, 580 μg, 600 μg, 700 μg, 800 g or 900 μg per dose. According to embodiments of the application, the toll-like receptor 4 agonist comprises 3D-(6-acyl) PHAD®.

As used herein, the term “CpG oligonucleotide”, “CpG oligodeoxynucleotide” or “CpG ODN” refers to an oligonucleotide comprising at least one CpG motif. As used herein, “oligonucleotide,” “oligodeoxynucleotide” or “ODN” refers to a polynucleotide formed from a plurality of linked nucleotide units. Such oligonucleotides can be obtained from existing nucleic acid sources or can be produced by synthetic methods. As used herein, the term “CpG motif” refers to a nucleotide sequence which contains unmethylated cytosine-phosphate-guanine (CpG) dinucleotides (i.e., a cytosine (C) followed by a guanine (G)) linked by a phosphate bond or a phosphodiester backbone or other internucleotide linkages.

According to particular embodiments, the CpG oligonucleotide is lipidated, i.e., conjugated (covalently linked) to a lipid moiety. As used herein, a “lipid moiety” refers to a moiety containing a lipophilic structure. Lipid moieties, such as an alkyl group, a fatty acid, a triglyceride, diglyceride, steroid, sphingolipid, glycolipid or a phospholipid, particularly a sterol such as cholesterol, or fatty acids, when attached to highly hydrophilic molecules, such as nucleic acids, can substantially enhance plasma protein binding and consequently circulation half-life of the hydrophilic molecules. In addition, binding to certain plasma proteins, such as lipoproteins, has been shown to increase uptake in specific tissues expressing the corresponding lipoprotein receptors (e.g., LDL-receptor HDL-receptor or the scavenger receptor SR-B1). In particular, a lipid moiety conjugated to the phosphopeptides and/or CpG oligonucleotide allows anchoring the said peptides and/or oligonucleotides into the membrane of a liposome via a hydrophobic moiety. According to particular embodiments, in view of the present disclosure, the CpG oligonucleotide can comprise any suitable internucleotide linkages.

As used herein, the term “internucleotide linkage” refers to a chemical linkage to join two nucleotides through their sugars consisting of a phosphorous atom and a charged or neutral group between adjacent nucleosides. Examples of internucleotide linkage include phosphodiester (po), phosphorothioate (ps), phosphorodithioate (ps2), methylphosphonate (mp), and methylphosphorothioate (rp). Phosphorotbioate, phosphorodithioate, methylphosphonate and methylphosphorothioate are stabilizing internucleotide linkages, while phosphodiester is a naturally-occurring internucleotide linkage. Oligonucleotide phosphorothioates are typically synthesized as a random racemic mixture of Rp and Sp phosphorothioate linkages.

In certain embodiments, an effective amount of liposomes further comprises a lipidated CpG oligonucleotide at an amount of 50 μg to 1250 μg, preferably 100 μg to 1000 μg, such as 150 μg to 800 μg, 150 to 900 μg, 125 μg to 950 μg or 150 μg to 850 μg per dose. For example, the effective amount of liposomes can comprise a lipidated CpG oligonucleotide at an amount of 50 μg, 100 μg, 150 μg, 200 μg, 250 μg, 300 μg, 350 μg, 400 μg, 450 μg, 500 μg, 550 μg, 600 Hg, 650 μg, 700 μg. 750 μg, 800 μg, 850 μg, 900 μg, 950 μg, 1000 μg, 1050 μg, 1100 μg, 1200 μg, or 1250 μg per dose. According to embodiments of the application, the lipidated CpG oligonucleotide is a CpG oligonucleotide comprising a nucleotide sequence of one of SEQ ID NOs: 18-22, preferably the lipidated CpG oligonucleotide is a CpG oligonucleotide comprising a nucleotide sequence of SEQ ID NO: 18. According to embodiments of the application, the lipidated CpG oligonucleotide is a CpG oligonucleotide comprising a nucleotide sequence of SEQ ID NO: 18 which has one or more phosphorothioate internucleotide linkages and is covalently linked to cholesterol via a linker comprising polyethylene glycol (PEG).

As used herein, the term “helper T-cell epitope” refers to a polypeptide comprising an epitope that is capable of recognition by a helper T-cell. Examples of helper T-cell epitopes include, but are not limited to, tetanus toxoid (e.g., the P2 and P30 epitopes, also named, respectively as T2 and T30), Hepatitis B surface antigen, cholera toxin B, diphtheria toxoid, measles virus F protein, Chlamydia trachomatis major outer membrane protein, Plasmodium falciparum circumsporozite T, P. falciparum CS antigen, Schistosoma mansoni triose phosphate isomerase, Bordetella pertussis, Clostridium tetani, Pertusaria trachythallina, Escherichia coli TraT, and Influenza virus hemagglutinin (HA).

According to other embodiments of the application, an effective amount of liposomes further comprises a helper T-cell epitope at an amount of 25 μg to 625 μg, preferably 75 μg to 550 μg, such as 75 μg to 450 μg, 80 μg to 540 μg, 82.5 μg to 535 μg, 85 μg to 530 μg, 87.5 μg to 525 μg, or 90 μg to 520 μg, per dose. For example, the effective amount of liposomes can comprise a helper T-cell epitope at an amount of 25 μg, 50 μg, 70 μg, 72.5 μg, 75 μg, 77.5 μg, 80 μg, 82.5 μg, 85 μg, 87.5 μg, 90 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 550 μg, 575 μg, 600 μg, or 625 μg per dose. According to embodiments of the application, the helper T-cell epitope is a T50 helper T-cell epitope consisting of the amino acid sequence of SEQ ID NO: 13, a T46 helper T-cell epitope consisting of the amino acid sequence of SEQ ID NO: 14, a T48 helper T-cell epitope consisting of the amino acid sequence of SEQ ID NO: 15, a T51 helper T-cell epitope consisting of the amino acid sequence of SEQ ID NO: 16, a T52 helper T-cell epitope consisting of the amino acid sequence of SEQ ID NO: 17, or a T57 helper T-cell epitope consisting of the amino acid sequence of SEQ ID NO: 44, preferably the helper T-cell epitope is a T50 helper T-cell epitope consisting of the amino acid sequence of SEQ ID NO: 13.

In certain embodiments, an effective amount of a liposome according to the application is administered to a subject in need of a treatment of preclinical AD repeatedly. The timing of administrations can vary significantly from once a week or a month, to once a year, to once a decade. A typical regimen consists of an immunization followed by booster injections at time intervals, such as 1 to 24-week intervals. Another regimen consists of an immunization followed by booster injections 1, 2, 4, 6, 8, 10 and 12 months later. Another regimen entails an injection every two months for life. Alternatively, booster injections can be on an irregular basis as indicated by monitoring of immune response. It is readily appreciated by those skilled in the art that the regimen for the priming and boosting administrations can be adjusted based on the measured immune responses after the administrations. For example, the boosting compositions are generally administered weeks or months after administration of the priming composition, for example, about 1 week, or 2 weeks, or 3 weeks, or 4 weeks, or 8 weeks, or 16 weeks, or 20 weeks, or 24 weeks, or 28 weeks, or 32 weeks, or 36 weeks, or 40 weeks, or 44 weeks, or 48 weeks, or 52 weeks, or 56 weeks, or 60 weeks, or 64 weeks, or 68 weeks, or 72 weeks, or 76 weeks, or one to two years after administration of the priming composition. According to particular aspects, one or more boosting immunizations can be administered. The antigens in the respective priming and boosting compositions, however many boosting compositions are employed, need not be identical, but should share antigenic determinants or be substantially similar to each other.

In some embodiments, after the initial administration on Day 1, the effective amount of the liposome is administered to the human subject repeatedly, e.g., at week 8, weeks 24, week 50, and every 26 weeks after week 50.

In certain embodiments, a method of the application further comprises detecting one or more biomarkers related to Alzheimer's disease before, during, and/or after the administering step. Biomarkers related to Alzheimer's disease include, for example but are not limited to, a positive PET scan on abnormal deposit(s) or plaques of beta-amyloid, a positive PET scan on pathological form of neurofibrillary tangles (NFT) of hyperphosphorylated tau, an altered level of beta-amyloid 42 or beta-amyloid 42/40, or an elevated level of tau and phosphorylated tau in CSF or plasma, an abnormal level of one or more other biomarkers in CSF or plasma, such as elevated plasma level of glial fibrillary acidic protein (GFAP) in plasma. For example, but not limited to, concentrations of CSF and/or plasma total tau (t-tau), p181−tau, p217+tau and other exploratory Tau-related biomarkers of interest (if applicable) can be measured. Tau proteins are normally associated with tubulin polymers to stabilize the microtubule system essential for normal neuronal function. However, tau is hyperphosphorylated in AD and is the main component in paired helical filaments, which then associate to form NFTs in the cytoplasm of many neurons. An elevation in levels of tau-related biomarkers in CSF or plasma is indicative of axonal injury and neurodegeneration downstream of the initiating pathology. The increase in hyperphosphorylated tau in CSF is relatively specific to AD and therefore distinguishes AD from other neurodegenerative disorders (Hampel 2004, Dialogues Clin Neurosci. 2004; 6:379-390, Cicognola et al., Alzheimers Res Ther. 2021; 13:38). Multiple sampling time points for CSF and plasma collection will permit an assessment over time of the occurrence of changes in CSF/plasma p217+tau levels, other AD biomarkers or additional downstream markers of neurodegeneration and their respective time courses. The sample collected up to Week 0 (pre-dose) can be considered the baseline sample.

In some embodiments, the human subject in need of a treatment of preclinical AD has pathological tau or a tau pathology as identified using a plasma phosphorylated tau immunoassay. In some embodiments, at the time of the initial treatment, the human subject has an elevated level of plasma phosphorylated tau, such as an elevated level of plasma phosphorylated tau 217, plasma phosphorylated tau181 or plasma phosphorylated tau231. The elevated level of plasma phosphorylated tau can be identified, for example, using a plasma phosphorylated tau217 immunoassay, a plasma phosphorylated tau181 immunoassay, and/or a plasma phosphorylated tau231 immunoassay. In some embodiments, at the time of the initial treatment, the human subject has an elevated level of plasma phosphorylated tau217. The elevated level of plasma phosphorylated tau217 can be identified, for example, using a plasma phosphorylated tau217 immunoassay. The presence of the tau pathology in the human subject can be confirmed using a positron emission tomography (PET) scan. Embodiments of the application also relate to a kit containing reagents (such as antibody against the phosphorylated tau) and instructions for the plasma phosphorylated tau immunoassay.

In certain embodiments, a method of the application further comprises administering to the subject one or more additional therapies in combination with the effective amount of the liposomes. In some embodiment of the invention, a liposome composition according to the invention, can be used in combination with a biologically active substance such as, for example, known compounds used in the medication of tauopathies and/or of amyloidosis.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

The invention can be described with reference to the following numbered embodiments:

### Embodiments Section A

Embodiment 1: A method of treating preclinical Alzheimer's disease in a human subject, comprising administering an effective amount of a liposome to the human subject, wherein the liposome comprises:
a. a tau peptide having an amino acid sequence selected from the group consisting of SEQ ID NOs: 2, 5, 9, and 12, wherein the tau peptide is presented on the surface of the liposome;
b. a helper T cell epitope comprising at least one amino acid sequence selected from the group consisting of: SEQ ID NOs: 23, 24, 25, and 26;
c. a lipidated CpG oligonucleotide, wherein the CpG oligonucleotide comprises one or more phosphorothioate internucleotide linkages, and wherein the CpG oligonucleotide is covalently linked to at least one cholesterol via a linker; and
d. monophosphoryl lipid A (MPLA).

Embodiment 2: A method of blocking the seeding activity of paired helical filaments (PHF) or enriched paired helical filaments (ePHF) and preventing the formation and/or accumulation of Tau aggregates in a human subject having preclinical AD, comprising administering an effective amount of a liposome to the human subject, wherein the liposome comprises:
a. a tau peptide having an amino acid sequence selected from the group consisting of SEQ ID NOs: 2, 5, 9, and 12, wherein the tau peptide is presented on the surface of the liposome;
b. a helper T cell epitope comprising at least one amino acid sequence selected from the group consisting of: SEQ ID NOs: 23, 24, 25, and 26;
c. a lipidated CpG oligonucleotide, wherein the CpG oligonucleotide comprises one or more phosphorothioate internucleotide linkages, and wherein the CpG oligonucleotide is covalently linked to at least one cholesterol via a linker; and
d. monophosphoryl lipid A (MPLA).

Embodiment 3: The method of Embodiment 1 or 2, wherein at the time of the initial treatment, the human subject has one or more of the following criteria: (i) a tau pathology, such as an elevated brain tau pathology defined as Braak 3 ROI SUVR>1.1 on a screening tau PET scan, (ii) no diagnosis of Alzheimer's Dementia or non-Alzheimer's Dementia or Mild Cognitive Impairment, (iii) a Clinical Dementia Rating (CDR) global score of 0, and (iv) a Mini-Mental State Examination (MMSE) score of 25 or more; preferably, at the time of the initial treatment, the human subject has a tau pathology, particularly an elevated brain tau pathology defined as Braak 3 ROI SUVR>1.1 on a screening tau PET scan, and one of more of the criteria of (ii)-(iv); more preferably, at the time of the initial treatment, the human subject has all of the criteria of (i)-(iv).

Embodiment 4: The method of any of the foregoing Embodiments, wherein at the time of the initial treatment, the human subject is 75 years of age or younger, or 50 or 55 years of age or older, particularly 55-75 years of age.

Embodiment 5: The method of any one of the foregoing Embodiments, wherein at the time of the initial treatment, the human subject has an MMSE score of 27 or more.

Embodiment 6: The method of any one of the foregoing Embodiments, wherein at the time of the initial treatment, the human subject has one or more biomarkers related to Alzheimer's disease.

Embodiment 7: The method of any one of the foregoing Embodiments, wherein at the time of the initial treatment, the human subject has an elevated plasma phosphorylated tau (p-tau) level, particularly an elevated p-tau217, an elevated p-tau181, and/or an elevated p-tau231.

Embodiment 8: The method of Embodiment 7, wherein at the time of the initial treatment, the human subject has (i) a tau pathology, such as an elevated brain tau pathology defined as Braak 3 ROI SUVR>1.1 on a screening tau PET scan, (ii) no diagnosis of Alzheimer's Dementia or non-Alzheimer's Dementia or Mild Cognitive Impairment, (iii) a Clinical Dementia Rating (CDR) global score of 0, and (iv) a Mini-Mental State Examination (MMSE) score of 25 or more, particularly an MMSE score of 27 or more.

Embodiment 9: The method of any of the foregoing Embodiments, wherein the liposome comprises:
a. the tau peptide having an amino acid sequence selected from the group consisting of SEQ ID NO: 28, 31, 35, and 38;
b. the lipidated CpG oligonucleotide having the nucleotide sequence selected from the group consisting of SEQ ID NO:18 to SEQ ID NO:22; and
c. the helper T cell epitope comprising the amino acid sequences of SEQ ID NO: 23, SEQ ID NO:24 and SEQ ID NO:25, wherein the amino acid sequences are covalently linked together, optionally via one or more linkers.

Embodiment 10: The method of any of the foregoing Embodiments, wherein the liposome comprises the helper T cell epitope comprising an amino acid sequence selected from the group consisting of SEQ ID NOs: 39, 40, 41, 42, and 43, or the helper T cell epitope comprising an amino acid sequence selected from the group consisting of SEQ ID NOs: 13, 14, 15, 16, 17, and 44.

Embodiment 11: The method of any of the foregoing Embodiments, wherein the liposome comprises:
a. the tau peptide comprising the amino acid sequence of SEQ ID NO: 2, wherein the tau peptide is presented on the surface of the liposome;
b. the helper T cell epitope having an amino acid sequence selected from the group consisting of SEQ ID NOs: 39, 40, 41, 42, and 43;
c. the lipidated CpG oligonucleotide having the nucleotide sequence selected from the group consisting of SEQ ID NO: 18 to SEQ ID NO:22, wherein the CpG oligonucleotide is covalently linked to at least one cholesterol via a linker; and
d. monophosphoryl lipid A (MPLA).

Embodiment 12: The method of any of the foregoing Embodiments, wherein the liposome comprises:
a. the tau peptide comprising the amino acid sequence of SEQ ID NO: 28, wherein the tau peptide is presented on the surface of the liposome;
b. the helper T cell epitope having an amino acid sequence selected from the group consisting of SEQ ID NOs: 13, 14 or 15;
c. the lipidated CpG oligonucleotide having the nucleotide sequence selected from the group consisting of SEQ ID NO:18, wherein the CpG oligonucleotide is covalently linked to at least one cholesterol via a linker; and
d. monophosphoryl lipid A (MPLA).

Embodiment 13: The method of any of the foregoing Embodiments, wherein the liposome further comprises one or more lipids selected from the group consisting of 1,2-dimyristoyl-sn-glycero-3-phosphocholine (DMPC), 1,2-dimyristoyl-sn-glycero-3-phosphoryl-3′-rac-glycerol (DMPG), and cholesterol.

Embodiment 14: The method of any of the foregoing Embodiments, wherein the effective amount of the liposome comprises 300 μg to 1800 μg per dose of the Tau peptide.

Embodiment 15: The method of Embodiment 14, wherein the effective amount of the liposome comprises 300-600 μg per dose, such as 300, 400, 500 or 600 μg per dose, or any amount in between, more particularly 300 μg per dose, of the Tau peptide.

Embodiment 16: The method of Embodiment 14, wherein the effective amount of the liposome comprises 700-1100 μg per dose, such as 700, 800, 900, 1000 or 1100 μg per dose, or any amount in between, more particularly 900 μg per dose, of the Tau peptide.

Embodiment 17: The method of Embodiment 14, wherein the effective amount of the liposome comprises 1200-1800 μg per dose, such as 1200, 1300, 1400, 1500, 1600, 1700, or 1800 μg per dose, or any amount in between, more particularly 1800 μg per dose, of the Tau peptide.

Embodiment 18: The method of any of the foregoing Embodiments, wherein the effective amount of the liposome is administered subcutaneously.

Embodiment 19: The method of any one of Embodiments 1-17, wherein the effective amount of the liposome is administered intramuscularly.

Embodiment 20: The method of any of the foregoing Embodiments, further comprising administering to the subject a second dose of the effective amount of liposome 4 to 12 weeks, such as 8 weeks, after the initial administration of the effective amount of liposome.

Embodiment 21: The method of Embodiment 20, further comprising administering to the subject a third dose of the effective amount of liposome 20 to 28 weeks, such as 24 weeks, after the initial administration of the effective amount of liposome.

Embodiment 22: The method of Embodiment 21, further comprising administering to the subject one or more doses of the effective amount of liposome once every 26 weeks after the third dose, such as 50 weeks, 76 weeks, 92 weeks, 118 weeks, 134 weeks, 160 weeks, 186 weeks, and/or 204 weeks after the initial administration of the effective amount of liposome.

Embodiment 23: The method of any of the foregoing Embodiments, further comprising detecting one or more Alzheimer's disease biomarkers before, during, and/or after the administering step.

Embodiment 24: The method of Embodiment 21, wherein the Alzheimer's disease biomarkers comprise an elevated plasma phosphorylated tau (p-tau) level, particularly an elevated p-tau217, an elevated p-tau181, and/or an elevated p-tau231.

Embodiment 25: The method of any one of the foregoing Embodiments, wherein the treatment prevents or delays onset of clinical symptoms of AD, such as onset of cognitive impairment and/or functional impairment related to AD.

Embodiment 26: The method of Embodiment 25, wherein the treatment prevents or delays decline, or reduces the rate of the decline, of the human subject's cognitive or brain function to thereby prevent or delay the onset of clinical symptoms of AD.

Embodiment 27: The method of any one of the foregoing Embodiments, wherein the treatment mitigates or lessens neuropathology, particularly tau pathology, related to AD in the human subject; more particularly, the treatment blocks the seeding activity of PHF or enriched PHF (ePHF) and prevents the formation and/or accumulation of pathological Tau (e.g., aggregated tau or neurofibrillary tangles).

Embodiment 28: The method of any one of the foregoing Embodiments, wherein the treatment prevents or delays progression of the preclinical AD into subsequent symptomatic stages of AD, preferably as assessed by one or more of (1) the Preclinical Alzheimer's Cognitive Composite-5 (PACC-5) total global score; (2) tau PET scans; and (3) other clinical outcomes, such as Mild Behavioral Impairment Checklist (MBI-C), Activities of Daily Living Prevention Instrument (ADCS-ADL-PI), Clinical Dementia Rating (CDR)-Global Score (GS) and CDR-sum of boxes scores (SB).

Embodiment 29: The method of any one of the foregoing Embodiments, wherein at the time of the initial treatment, the human subject has an elevated level of plasma phosphorylated tau, such as an elevated level of plasma phosphorylated tau 217, plasma phosphorylated tau181 or plasma phosphorylated tau231.

Embodiment 30: The method of Embodiment 29, wherein the elevated level of plasma phosphorylated tau is identified using a plasma phosphorylated tau immunoassay, such as a plasma phosphorylated tau 217 immunoassay, a plasma phosphorylated tau181 immunoassay, and/or a plasma phosphorylated tau231 immunoassay.

Embodiment 31: The method of any one of Embodiments 1-28, wherein at the time of the initial treatment, the human subject has an elevated level of plasma phosphorylated tau 217.

Embodiment 32: The method of Embodiment 31, wherein the elevated level of plasma phosphorylated tau 217 is identified using a plasma phosphorylated tau 217 immunoassay.

Embodiment 33: The method of any one of Embodiment 29-32, wherein the presence of the tau pathology in the human subject is confirmed using a positron emission tomography (PET) scan.

### Embodiments Section B

Embodiment 34. A method of treating preclinical Alzheimer's disease in a human subject in need thereof, comprising intramuscularly administering an effective amount of a liposome to the human subject, wherein the liposome comprises:
(i) a tau peptide having the amino acid sequence selected from the group consisting of SEQ ID NOs: 1-12, wherein the tau peptide is presented on the surface of the liposome;
(ii) a helper T cell epitope comprising at least one amino acid sequence selected from the group consisting of: SEQ ID NOs: 23, 24, 25, and 26;
(iii) a lipidated CpG oligonucleotide, wherein the CpG oligonucleotide comprises one or more phosphorothioate internucleotide linkages, and wherein the CpG oligonucleotide is covalently linked to at least one cholesterol via a linker; and
(iv) monophosphoryl lipid A (MPLA), wherein the human subject has a tau pathology and has no cognitive impairment.

Embodiment 35: The method of Embodiment 34, wherein the tau peptide has the amino acid sequence of SEQ ID NO: 2, 5, 9 or 12.

Embodiment 36: The method of Embodiment 35, wherein the liposome comprises:
(i) the tau peptide having the amino acid sequence of SEQ ID NO: 2, 5, 9 or 12, wherein the tau peptide is presented on the surface of the liposome;
(ii) the helper T cell epitope comprising at least one amino acid sequence selected from the group consisting of: SEQ ID NOs: 23, 24, 25, and 26;
(iii) the lipidated CpG oligonucleotide, wherein the CpG oligonucleotide comprises one or more phosphorothioate internucleotide linkages, and wherein the CpG oligonucleotide is covalently linked to at least one cholesterol via a linker; and
(iv) monophosphoryl lipid A (MPLA).

Embodiment 37: The method of Embodiment 36, wherein
a. the tau peptide has an amino acid sequence selected from the group consisting of SEQ ID NO: 28, 31, 35, and 38;
b. the helper T cell epitope comprises the amino acid sequences of SEQ ID NO: 23, SEQ ID NO: 24 and SEQ ID NO: 25, wherein the amino acid sequences are covalently linked together, optionally via one or more linkers; and
c. the lipidated CpG oligonucleotide has the nucleotide sequence selected from the group consisting of SEQ ID NO: 18 to SEQ ID NO: 22.

Embodiment 38: The method of Embodiment 36 or 37, wherein the helper T cell epitope comprises an amino acid sequence selected from the group consisting of SEQ ID NOs: 39, 40, 41, 42, 43, 13, 14, 15, 16, 17, and 44.

Embodiment 39: The method of any of Embodiments 34 to 38, wherein the tau peptide comprises the amino acid sequence of SEQ ID NO: 2.

Embodiment 40: The method of Embodiment 39, wherein the liposome comprises:
a. the tau peptide comprising the amino acid sequence of SEQ ID NO: 2;
b. the helper T cell epitope having an amino acid sequence selected from the group consisting of SEQ ID NOs: 39, 40, 41, 42, 43, 13, 14, 15, 16, 17, and 44.
c. the lipidated CpG oligonucleotide having the nucleotide sequence selected from the group consisting of SEQ ID NO: 18 to SEQ ID NO: 22, wherein the CpG oligonucleotide is covalently linked to at least one cholesterol via a linker; and
d. monophosphoryl lipid A (MPLA).

Embodiment 41: The method of Embodiment 40, wherein:
a. the tau peptide has the amino acid sequence of SEQ ID NO: 28;
b. the helper T cell epitope having an amino acid sequence selected from the group consisting of SEQ ID NOs: 39, 40, 41, 42, and 43; and
c. the lipidated CpG oligonucleotide having a nucleotide sequence selected from the group consisting of SEQ ID NO: 18 to SEQ ID NO:22, wherein the CpG oligonucleotide is covalently linked to at least one cholesterol via a linker.

Embodiment 42: The method of Embodiment 41, wherein:
a. the tau peptide comprises the amino acid sequence of SEQ ID NO: 28;
b. the helper T cell epitope has an amino acid sequence selected from the group consisting of SEQ ID NOs: 13, 14 or 15; and
c. the lipidated CpG oligonucleotide having the nucleotide sequence selected from the group consisting of SEQ ID NO: 18.

Embodiment 43: The method of any one of Embodiments 34 to 42, wherein the liposome further comprises one or more lipids selected from the group consisting of 1,2-dimyristoyl-sn-glycero-3-phosphocholine (DMPC), 1,2-dimyristoyl-sn-glycero-3-phosphoryl-3′-rac-glycerol (DMPG), and cholesterol.

Embodiment 44: The method of any one of Embodiments 34 to 43, wherein the human subject has a tau pathology as measured by an elevated plasma level of p-tau217, p-tau181, and/or p-tau231, and/or by a tau positron emission tomography (PET) scan at the initial administration of the effective amount of the liposome.

Embodiment 45: The method of any one of Embodiments 34 to 44, wherein the human subject has no diagnosis of Alzheimer's Dementia or non-Alzheimer's Dementia or Mild Cognitive Impairment, and has at least one of (i) an elevated brain tau pathology defined as Braak III region of interest standardized uptake value ratio (Braak 3 ROI SUVR)>1.1 on a screening tau PET scan, (ii) a Clinical Dementia Rating (CDR) global score of 0, and/or (iii) a Mini-Mental State Examination (MMSE) score of 25 or more, at the initial administration of the effective amount of the liposome.

Embodiment 46: The method of Embodiment 45, the human subject has (i) an elevated brain tau pathology defined as Braak III region of interest standardized uptake value ratio (Braak 3 ROI SUVR)>1.1 on a screening tau PET scan, (ii) a CDR global score of 0, and (iii) a MMSE score of 25 or more, preferably 27 or more, at the initial administration of the effective amount of the liposome.

Embodiment 46a: The method of Embodiment 45, the human subject has (i) an elevated brain tau pathology defined as Braak III region of interest standardized uptake value ratio (Braak 3 ROI SUVR)>1.1 on a screening tau PET scan, (ii) a CDR global score of 0, and (iii) a MMSE score of 25 or more, at the initial administration of the effective amount of the liposome.

Embodiment 46b: The method of Embodiment 45, the human subject has (i) an elevated brain tau pathology defined as Braak III region of interest standardized uptake value ratio (Braak 3 ROI SUVR)>1.1 on a screening tau PET scan, (ii) a CDR global score of 0, and (iii) a MMSE score of 27 or more, at the initial administration of the effective amount of the liposome.

Embodiment 47: The method of any one of Embodiments 34 to 46b, wherein the human subject is 50-75 years of age at the initial administration of the effective amount of the liposome.

Embodiment 48: The method of any one of Embodiments 34 to 47, wherein the effective amount of the liposome comprises 300 μg to 1800 μg per dose of the tau peptide.

Embodiment 49: The method of any one of Embodiments 34 to 48, wherein the effective amount of the liposome comprises 900 μg per dose of the tau peptide.

Embodiment 50: The method of any one of Embodiments 34 to 49, wherein the effective amount of the liposome is administered to the human subject once every 6 months for a period of at least 1 year.

Embodiment 51: The method of Embodiment 50, further comprising:
(a) intramuscularly administering the effective amount of the liposome to the human subject 24 weeks before initiating the once every 6 months administrations; and
(b) intramuscularly administering the effective amount of the liposome to the human subject 16 weeks before initiating the once every 6 months administrations.

Embodiment 52: The method of Embodiment 50 or 51, wherein the effective amount of the liposome is administered to the human subject once every 6 months for a period of at least 1.5 years, at least 2 years, at least 2.5 years, at least 3 years, at least 3.5 years or at least 4 years.

Embodiment 53: The method of any one of Embodiments 34 to 52, comprising:
(a) intramuscularly administering the effective amount of a liposome to the human subject at week 0 of the treatment;
(b) intramuscularly administering the effective amount of the liposome to the human subject at week 8 of the treatment; and
(c) intramuscularly administering the effective amount of the liposome to the human subject once every 6 months for a period of at least 1 year, wherein the period starts at week 24 of the treatment.

Embodiment 54: The method of any one of Embodiments 34 to 53, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

### Embodiments Section C

Embodiment 55: A method of treating preclinical Alzheimer's disease in a human subject in need thereof, comprising intramuscularly administering an effective amount of a liposome to the human subject, wherein the liposome comprises:
(i) a tau peptide having the amino acid sequence of SEQ ID NO: 2, wherein the tau peptide is presented on the surface of the liposome;
(ii) a helper T cell epitope comprising at least one amino acid sequence selected from the group consisting of: SEQ ID NOs: 23, 24, 25, and 26;
(iii) a lipidated CpG oligonucleotide, wherein the CpG oligonucleotide comprises one or more phosphorothioate internucleotide linkages, and wherein the CpG oligonucleotide is covalently linked to at least one cholesterol via a linker; and
(iv) monophosphoryl lipid A (MPLA), wherein the human subject has a tau pathology and no cognitive impairment.

Embodiment 56: The method of Embodiment 55, wherein the human subject has a tau pathology as measured by an elevated plasma level of p-tau217, p-tau181, and/or p-tau231, and/or by a tau positron emission tomography (PET) scan at the initial administration of the effective amount of the liposome.

Embodiment 57: The method of Embodiment 56, wherein the human subject has no diagnosis of Alzheimer's Dementia or non-Alzheimer's Dementia or Mild Cognitive Impairment, and has at least one of (i) an elevated brain tau pathology defined as Braak III region of interest standardized uptake value ratio (Braak 3 ROI SUVR)>1.1 on a screening tau PET scan, (ii) a Clinical Dementia Rating (CDR) global score of 0, and/or (iii) a Mini-Mental State Examination (MMSE) score of 25 or more, at the initial administration of the effective amount of the liposome.

Embodiment 58: The method of Embodiment 55, wherein the human subject is 50-75 years of age at the initial administration of the effective amount of the liposome.

Embodiment 59: The method of Embodiment 55, wherein the effective amount of the liposome comprises 300 μg to 1800 μg per dose of the tau peptide.

Embodiment 60: The method of Embodiment 55, wherein the effective amount of the liposome comprises 900 μg per dose of the tau peptide.

Embodiment 61: The method of Embodiment 55, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

Embodiment 62: A method of treating preclinical Alzheimer's disease in a human subject in need thereof, comprising intramuscularly administering an effective amount of a liposome to the human subject once every 6 months for a period of at least 1 year, wherein the liposome comprises:
(i) a tau peptide having the amino acid sequence of SEQ ID NO: 2, wherein the tau peptide is presented on the surface of the liposome;
(ii) a helper T cell epitope comprising at least one amino acid sequence selected from the group consisting of: SEQ ID NOs: 23, 24, 25, and 26;
(iii) a lipidated CpG oligonucleotide, wherein the CpG oligonucleotide comprises one or more phosphorothioate internucleotide linkages, and wherein the CpG oligonucleotide is covalently linked to at least one cholesterol via a linker; and
(iv) monophosphoryl lipid A (MPLA);
wherein the human subject has a tau pathology and no cognitive impairment.

Embodiment 62a: A method of treating preclinical Alzheimer's disease in a human subject in need thereof, comprising intramuscularly administering an effective amount of a liposome to the human subject once every 6 months for a period of at least 1 year, wherein the liposome comprises:
(i) a tau peptide having the amino acid sequence of SEQ ID NO: 2, wherein the tau peptide is presented on the surface of the liposome;
(ii) a helper T cell epitope comprising at least one amino acid sequence selected from the group consisting of: SEQ ID NOs: 23, 24, 25, and 26;
(iii) a lipidated CpG oligonucleotide, wherein the CpG oligonucleotide comprises one or more phosphorothioate internucleotide linkages, and wherein the CpG oligonucleotide is covalently linked to at least one cholesterol via a linker; and
(iv) monophosphoryl lipid A (MPLA).

Embodiment 63: The method of Embodiment 62, wherein the human subject has a tau pathology as measured by an elevated plasma level of p-tau217, p-tau181, and/or p-tau231, and/or by a tau positron emission tomography (PET) scan at the initial administration of the effective amount of the liposome.

Embodiment 64: The method of Embodiment 63, wherein the human subject has no diagnosis of Alzheimer's Dementia or non-Alzheimer's Dementia or Mild Cognitive Impairment, and has at least one of (i) an elevated brain tau pathology defined as Braak III region of interest standardized uptake value ratio (Braak 3 ROI SUVR)>1.1 on a screening tau PET scan, (ii) a Clinical Dementia Rating (CDR) global score of 0, and/or (iii) a Mini-Mental State Examination (MMSE) score of 25 or more, at the initial administration of the effective amount of the liposome.

Embodiment 65: The method of Embodiment 62 or 62a, wherein the human subject is 50-75 years of age at the initial administration of the effective amount of the liposome.

Embodiment 66: The method of Embodiment 62 or 62a, wherein the effective amount of the liposome comprises 300 μg to 1800 μg per dose of the tau peptide.

Embodiment 67: The method of Embodiment 66, wherein the effective amount of the liposome comprises 900 μg per dose of the tau peptide.

Embodiment 68: The method of Embodiment 62 or 62a, further comprising:
(a) intramuscularly administering the effective amount of the liposome to the human subject 24 weeks before initiating the once every 6 months administrations; and
(b) intramuscularly administering the effective amount of the liposome to the human subject 16 weeks before initiating the once every 6 months administrations.

Embodiment 69: The method of Embodiment 62 or 62a, wherein the effective amount of the liposome is administered to the human subject once every 6 months for a period of at least 1.5 years, at least 2 years, at least 2.5 years, at least 3 years, at least 3.5 years or at least 4 years.

Embodiment 70: The method of Embodiment 62 or 62a, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

Embodiment 71: A method of treating preclinical Alzheimer's disease in a human subject, comprising:
(a) intramuscularly administering an effective amount of a liposome to the human subject at week 0 of the treatment;
(b) intramuscularly administering the effective amount of the liposome to the human subject at week 8 of the treatment; and
(c) intramuscularly administering the effective amount of the liposome to the human subject once every 6 months for a period of at least 1 year, wherein the period starts at week 24 of the treatment,
wherein the liposome comprises:
(i) a tau peptide having the amino acid sequence of SEQ ID NO: 28, wherein the tau peptide is presented on the surface of the liposome;
(ii) a helper T cell epitope having an amino acid sequence selected from the group consisting of SEQ ID NOs: 39, 40, 41, 42, and 43;
(iii) a lipidated CpG oligonucleotide having a nucleotide sequence selected from the group consisting of SEQ ID NO: 18 to SEQ ID NO:22, wherein the CpG oligonucleotide is covalently linked to at least one cholesterol via a linker; and (iv) monophosphoryl lipid A (MPLA),
wherein the human subject has a tau pathology and no cognitive impairment.

Embodiment 72: The method of Embodiment 71, wherein the human subject has a tau pathology as measured by an elevated plasma level of p-tau217, p-tau181, and/or p-tau231, and/or by a tau PET scan, at the initial administration of the effective amount of the liposome.

Embodiment 73: The method of Embodiment 72, wherein at week 0, the human subject has no diagnosis of Alzheimer's Dementia or non-Alzheimer's Dementia or Mild Cognitive Impairment, and has at least one of (i) an elevated brain tau pathology defined as Braak III region of interest standardized uptake value ratio (Braak 3 ROI SUVR)>1.1 on a screening tau PET scan, (ii) a Clinical Dementia Rating (CDR) global score of 0, and/or (iii) a Mini-Mental State Examination (MMSE) score of 27 or more, at the initial administration of the effective amount of the liposome.

Embodiment 74: The method of Embodiment 71, wherein the human subject is 50-75 years of age at week 0.

Embodiment 75: The method of Embodiment 74, wherein the effective amount of the liposome comprises 300 μg to 1800 μg per dose of the tau peptide.

Embodiment 76: The method of Embodiment 75, wherein the effective amount of the liposome comprises 900 μg per dose of the tau peptide.

Embodiment 77: The method of Embodiment 71, wherein the effective amount of the liposome is administered to the human subject once every 6 months for a period of at least 1.5 years, at least 2 years, at least 2.5 years, at least 3 years, at least 3.5 years or at least 4 years.

Embodiment 78: The method of Embodiment 71, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

Embodiment 79: A method of treating preclinical Alzheimer's disease in a human subject, comprising:
(a) intramuscularly administering an effective amount of a liposome to the human subject at week 0 of the treatment;
(b) intramuscularly administering the effective amount of the liposome to the human subject at week 8 of the treatment; and
(c) intramuscularly administering the effective amount of the liposome to the human subject once every 6 months for a period of at least 1 year, wherein the period starts at week 24 of the treatment,
wherein the liposome comprises:
(i) a tau peptide comprising the amino acid sequence of SEQ ID NO: 28, wherein the tau peptide is presented on the surface of the liposome;
(ii) a helper T cell epitope having an amino acid sequence selected from the group consisting of SEQ ID NOs: 13, 14 or 15;
(iii) a lipidated CpG oligonucleotide having the nucleotide sequence selected from the group consisting of SEQ ID NO: 18, wherein the CpG oligonucleotide is covalently linked to at least one cholesterol via a linker; and
(iv) monophosphoryl lipid A (MPLA).
wherein the human subject is 50-75 years of age and has a tau pathology as measured by an elevated plasma level of p-tau217 and a tau PET scan at week 0 and has no cognitive impairment.

Embodiment 80: The method of Embodiment 79, wherein at week 0, the human subject has no diagnosis of Alzheimer's Dementia or non-Alzheimer's Dementia or Mild Cognitive Impairment, and has (i) an elevated brain tau pathology defined as Braak III region of interest standardized uptake value ratio (Braak 3 ROI SUVR)>1.1 on a screening tau PET scan, (ii) a CDR global score of 0, and (iii) a MMSE score of 27 or more.

Embodiment 81: The method of Embodiment 79, wherein the effective amount of the liposome comprises 300 μg to 1800 μg per dose of the tau peptide.

Embodiment 82: The method of Embodiment 81, wherein the effective amount of the liposome comprises 900 μg per dose of the tau peptide.

Embodiment 83: The method of Embodiment 79, wherein the effective amount of the liposome is administered to the human subject once every 6 months for a period of at least 1.5 years, at least 2 years, at least 2.5 years, at least 3 years, at least 3.5 years or at least 4 years.

Embodiment 84: The method of Embodiment 79, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

### EXAMPLES

The following examples are provided to further describe some of the embodiments disclosed herein. The examples are intended to illustrate, not to limit, the disclosed embodiments.

Studies, such as those described infra, demonstrate that antibodies generated after immunization with ACI-35.030 specifically interfered with Tau seeding in neurons, reduced the seeding activity of ePHF and prevented the formation and accumulation of Tau aggregates inside the neurons, thereby blocking the spread of extracellular pTau aggregates between brain regions. The studies also demonstrate that subsequent immunizations with ACI-35.030 generated antibodies with increased capacity to block the seeding activity of ePHF. A phase 2b RETAIN study, the first clinical trial in preclinical AD with a tau targeting active immunotherapy, is launched to assess efficacy, safety and immunogenicity of ACI-35.030 in preclinical AD population.

### Example 1. Antibodies Generated after Immunization of Rhesus Macaques with ACI-35.030 Specifically Reduce the Seeding Activity of ePHF Thereby Preventing Accumulation of Tau Aggregates in Primary Rat Cortical Neurons

All animal experiments were approved and performed in accordance with local legislation on animal experiments. Rhesus macaques (Macaca mulatta) were obtained from Kunming Biomed International Ltd, China. Animals were 2.5 to 5.1 years old at the start of immunization, and their minimum weight was 3.1 kg. A detailed clinical examination was performed prior to initiation of the treatment and weekly thereafter. Moreover, macaques were observed twice per day, and clinical signs were recorded. Groups of Rhesus macaques (n=3 males and 3 females per group) were immunized intramuscularly at day 1, 29 and 85 with i) 2400 μg of acetate tetrapalmitoylated phosphorylated tau peptide of SEQ ID NO: 2 per dose of ACI-35.030 active immunotherapy; or ii) 0 μg of acetate tetrapalmitoylated phosphorylated tau peptide of SEQ ID NO: 2 per dose of ACI-35E.030 placebo (liposomal formulation containing 3D-(6-acyl) PHAD®, lipidated CpG oligonucleotide CpG 2006 and T-cell peptide T50). Blood sampling was performed before the initial immunization (pre-dose [Day-14]) and after three immunizations (post-dose [Day 99]) and the serum was isolated.

The capacity of antibodies generated two weeks after the third immunization (Day 99) to inhibit the seeding activity of enriched paired helical filaments (ePHFs) extracted from a human Alzheimer's Disease brain was determined using primary rat cortical neurons. Isolation of ePHF was carried out based on the procedure described by Rostagno and Ghiso (Rostagno et al., 2009). Primary rat cortical neurons were incubated for two weeks with ePHF in the absence or presence of antibodies (using pooled pre-dose [Day-14] or post-dose [Day 99] monkey serum from all six individual Rhesus macaques per group, respectively) and formation of de-novo rat Tau aggregates inside cultured cells was determined by immunocytochemistry.

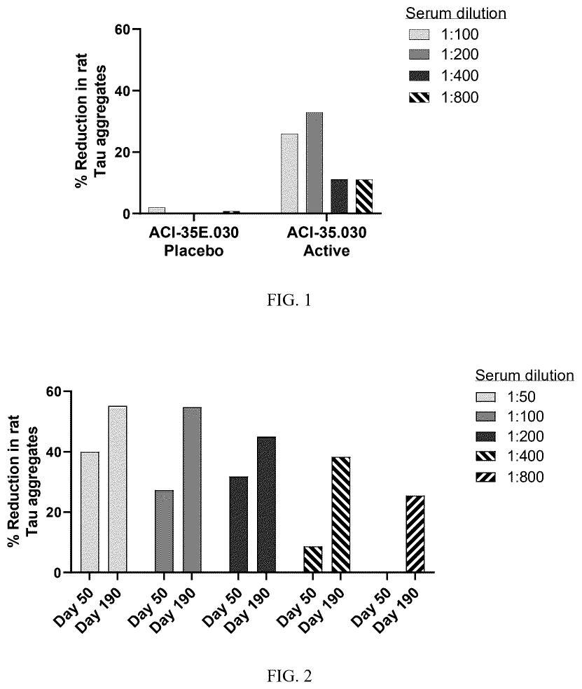

FIG. 1 shows the percent reduction of rat Tau aggregates present in primary rat cortical neurons after incubation with ePHF in the presence of post-dose (Day 99) as compared to pre-dose (Day-14) monkey serum pool. Whereas monkeys immunized at Day 1, 29 and 85 with ACI-35.030 active immunotherapy generated antibodies that decreased the formation of de-novo rat Tau aggregates inside primary rat cortical neurons, antibodies produced in monkeys immunized with ACI-35E.030 placebo did not show any effect. This data suggests that antibodies generated after immunization with ACI-35.030 can block the seeding activity of ePHF and further prevent the formation and accumulation of Tau aggregates inside the cells.

### Example 2. Efficacy of Antibodies Generated after Immunization of Rhesus Monkeys with ACI-35.030 to Reduce the Seeding Activity of ePHF Increases with Subsequent Immunizations

All animal experiments were approved and performed in accordance with local legislation on animal experiments. Rhesus macaques (Macaca mulatta) were obtained from Kunming Biomed International Ltd, China. Animals were two to five years old at the start of immunization, and their minimum weight was 3.6 kg. A detailed clinical examination was performed prior to initiation of the treatment and weekly thereafter. Moreover, macaques were observed twice per day, and clinical signs were recorded. A group of six Rhesus macaques (n=3 males and 3 females) were immunized subcutaneously at day 1, 29, 85 and 169 with 1800 μg of acetate tetrapalmitoylated phosphorylated tau peptide of SEQ ID NO: 2 per dose of ACI 35.030 active immunotherapy. Blood sampling was performed before the initial immunization (pre-dose [Day-63]) and after two and four immunizations (post-dose [Day 50] and post-dose [Day 190], respectively) and the serum was isolated.

The capacity of antibodies generated three weeks after the second or fourth immunization (Day 50 and Day 190, respectively) to inhibit the seeding activity of ePHF was determined using primary rat cortical neurons. Primary rat cortical neurons were incubated for two weeks with ePHF in the absence or presence of antibodies (using pooled pre-dose [Day-63] or post-dose [Day 50 or Day 190] monkey serum from all six individual Rhesus macaques, respectively) and formation of de-novo rat Tau aggregates inside cultured cells was determined by immunocytochemistry.

FIG. 2 shows the percent reduction of rat Tau aggregates present in primary rat cortical neurons after incubation with ePHF in the presence of post-dose (Day 50 or Day 190) as compared to pre-dose (Day-63) monkey serum pool. Monkeys immunized twice (at Day 1 and 29) as well as four times (at Day 1, 29, 85 and 169) with ACI-35.030 active immunotherapy generated antibodies that decreased the formation of de-novo rat Tau aggregates inside primary rat cortical neurons. A more pronounced reduction in rat Tau aggregates was seen after four (Day 190) as compared to after two (Day 50) immunizations. This data suggests that the capacity of antibodies generated after immunization with ACI-35.030 to block the seeding activity of ePHF increases with subsequent immunizations, indicating a maturation of the antibody response over time.

### Example 3. Functional Testing of Antibodies Generated after Immunization

Another functional assay was conducted to evaluate the capacity of antibodies generated by Tau active immunotherapy to interfere with Tau seeding in neurons. AD-Tau seeds extracted from Alzheimer's disease (AD) brain—Braak VI (according to Braak et al., Acta Neuropathol, 1991. 82(4): p. 239-59) are considered the most relevant representation of the extracellular Tau species targeted by Tau active immunotherapy. These AD-Tau seeds can enter rat cortical neurons and induce the aggregation of endogenous rat Tau (Guo, et al., J Exp Med, 2016. 213(12): p. 2635-2654). Polyclonal serum from rhesus monkeys treated with ACI-35.030 was assessed in the functional assay for its ability to inhibit Tau seeding by binding the AD-Tau seeds and prevent neuronal uptake and aggregation induction in the cells.

Polyclonal serum collected from 6 Rhesus monkeys immunized with 2 subcutaneous (SC) doses of ACI-35.030 (NHP serum d50, 50 days after study start) or 4 SC doses of JNJ-64042056 (NHP serum d190, 190 days after study start) was assessed for its capacity to inhibit Tau aggregation and compared to serum collected from the same animals prior to the first immunization (NHP serum d-14). Serum was analyzed in several different dilutions (1/50, 1/100, 1/200, 1/400, 1/800) in 3 independent experiments (including d-14 vs d190, including d-14 vs d50 vs d190).

Serum samples were co-incubated with AD-Tau seeds for 1 h at room temperature (RT) before adding the mixture to the neurons to incubate for 4 hours after which medium is removed and fresh medium is added to the cells. Neurons treated with AD-Tau seeds alone and neurons without the induction of aggregation by seeding were included as respectively high and low controls.

At day in vitro (DIV) 15, cells were lysed and the lysates were analyzed using MSD for the amount of aggregated rodent Tau and the amount of total rodent alpha-synuclein (aSyn). The aggregated rodent Tau assay used mTau8 [Rodrigues Martins, et al, J Alzheimers Dis, 2023. 93 (1): p. 151-167] as coating and detection antibody. The total aSyn MSD was used as normalization factor to control for possible differences in number of cells per well due to plate variation. Ratios of aggregated rodent Tau over total rodent aSyn were calculated per well and expressed as % of remaining seeding relative to the AD-Tau seeds only condition, for which the average is put at 100%. Statistical analysis was performed on the % remaining seeding as calculated for each condition in all three separate experiments.

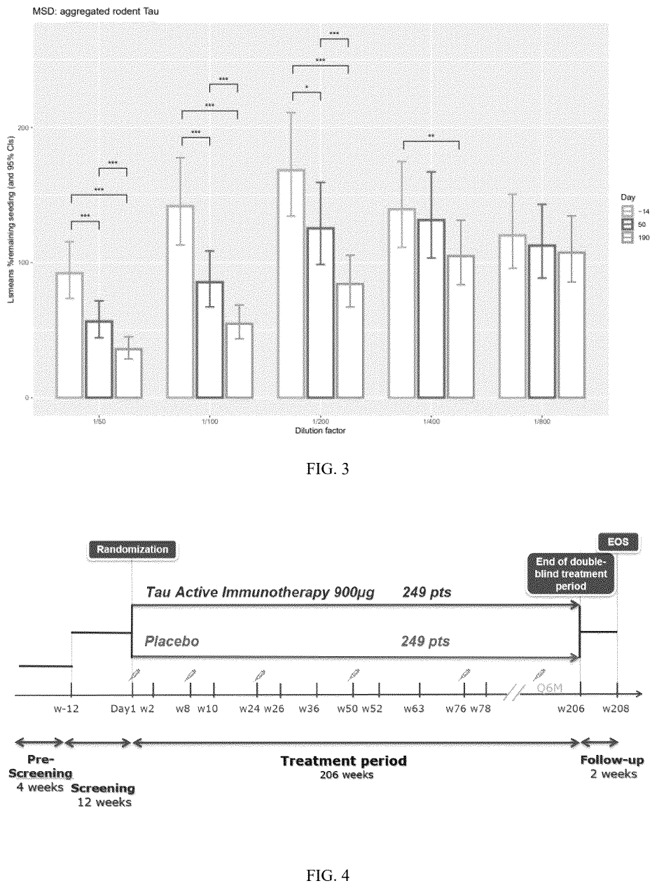

Results showed that co-incubation of AD-Tau seeds with NHP serum d50 significantly reduced the induction of rodent Tau aggregation inside the cells by 39% as measured at dilution 1/50, 40% as measured at dilution 1/100 or 26% as measured at dilution 1/200 compared to d-14 serum (FIG. 3). NHP serum d190 significantly reduced the amount of rodent Tau aggregates inside the cells by 61% as measured at dilution 1/50, 61% as measured at dilution 1/100, 50% as measured at dilution 1/200 or 25% at dilution 1/400 compared to d-14 serum (FIG. 3). A total of 3 independent experiments were analyzed.

The results demonstrated that on average, the antibodies generated in NHP after 2 and 4 doses of ACI-35.030 (respectively day 50 and day 190) were able to inhibit Tau aggregation when compared to pre-treatment samples (day-14). Most consistent inhibition of Tau aggregation was obtained for serum dilutions 1/50, 1/100 and 1/200, with higher dilutions progressively losing capacity to reduce Tau aggregation. This finding is consistent with the notion that antibody concentration is decreasing with increasing serum dilutions, which decreases the number of available antibodies to bind aggregated Tau seeds and therefore reduces efficiency of interfering with Tau seeds uptake.

### Example 4. A Phase 2b RETAIN Study of a Phosphorylated Tau Targeted Active Immunotherapy in Participants with Preclinical Alzheimer's Disease

There is consensus that the genesis of AD pathology predates dementia onset by over 20 years (Bateman 2012, Engl J Med. 2012; 367:795-804; Jack 2013, Lancet Neurol. 2013 February; 12 (2): 207-216), presenting an opportunity for prophylactic/preventive strategies aimed at disease course modification before dementia onset and even prior to the appearance of clinical symptoms.

Preclinical AD with elevated brain amyloid and tau (A+T+) specifically is considered a marker of advanced AD pathological changes and is strongly associated with near-term (i.e., 3-5 years) cognitive decline in cognitively unimpaired individuals and is therefore of high clinical relevance (Ossenkoppele 2022, Nat Med. 2022; 28 (11): 2381-2387). For example, about 50% of A+T+ preclinical AD individuals transition to Mild Cognitive Impairment (MCI) within 3.5 years vs. only about 4-10% of A+T− preclinical AD individuals.

Hence, evidence of advanced AD pathology provided by amyloid and tau positive positron emission tomography (PET) is strongly associated with near-term clinical progression in initially cognitively unimpaired individuals. This disease stage is associated with a dynamic change of tauopathy proximate to symptom onset and hence may be the optimal stage to measure a treatment effect and the optimal window of interception with antibodies targeting the tau seeds.

The multi-center randomized, placebo-controlled, double-blind, parallel-group study assesses the efficacy (including cognitive decline), safety and immunogenicity of an active immunotherapy ACI-35.030 targeting pathological tau over approximately 48 months treatment in participants with preclinical AD, and the last injection of the immunotherapy can be administered earlier than 48 months considering the long-lasting duration of the immune response induced by the immunotherapy. ACI-35.030 is designed to stimulate the immune system to produce antibodies against pTau.

The RETAIN study is the first interventional, double blind, clinical trial using an anti-tau active immunotherapy in preclinical AD, such as sporadic preclinical AD. This study primarily aims to investigate the effect of ACI-35.030 versus placebo on cognitive decline, as assessed by Preclinical AD Cognitive Composite 5 (PACC-5) total scores. The ease of administration (e.g., intramuscular (IM) injection) and low-burden dosing regimen (e.g., once every 1, 2, 3, 4, 5, or 6 months over the long-term) makes ACI-35.030 a candidate for large scale intervention in people with the asymptomatic stage of the disease.

This study consists of:
a pre-screening phase (approximately 4 weeks)
a screening period (up to 13 weeks prior to randomization)
a double-blind treatment period (approximately 48 months)
a post-treatment follow-up period (2 weeks)

The primary objective of this study is to investigate the effect of ACI-35.030 versus placebo on cognitive decline, as assessed by Preclinical AD Cognitive Composite 5 (PACC-5) total scores. The primary efficacy endpoint is change from baseline in PACC-5 total score at Week 206. A key secondary objective is to assess the effect of ACI-35.030 on the propagation and/or accumulation of tau pathology compared with placebo, as measured by tau PET in the Tau Naïve Composite region of interest. Planned secondary endpoints include the CDR-GS (time-to-event), CDR-SB, MBI-C, ADCS-ADL-PI, volumetric MRI, and CSF/plasma biomarkers.

In addition to the primary and key secondary objectives, additional secondary and tertiary objectives, such as those shown in Table 1 below, will also be measured.

TABLE 1
Study Objectives, Endpoints and/or Estimands

| Objectives | Endpoints |
|---|---|
| Primary | |
| To investigate the effect of ACI-35.030 | Change from baseline on PACC-5 total |
| versus placebo on cognitive decline, as | scores at Week 206 |
| assessed by Preclinical AD Cognitive | |
| Composite 5 (PACC-5) total scores | |
| Key Secondary | |
| To assess the effect of ACI-35.030 on the | Change from baseline in brain tau burden, as |
| propagation and/or accumulation of tau | measured by tau PET in the Tau Naïve |
| pathology compared with placebo, as | Composite region of interest (ROI) at Week |
| measured by tau PET in the Tau Naive | 206, 154 and 102 |
| Composite region of interest | |
| Secondary | |
| To investigate the effects of ACI-35.030 | Change from baseline on PACC-5 individual |
| versus placebo on cognitive decline, as | domain scores |
| assessed by PACC-5 individual domain scores | |
| To investigate the effect of ACI-35.030 versus | Time to event of clinical progression |
| placebo on clinical progression as measured | (defined as two consecutive increases from |
| by the Clinical Dementia Rating-Global Score | baseline CDR-GS of 0 to postbaseline CDR- |
| (CDR-GS) | GS of ≥0.5) |
| To investigate the effect of ACI-35.030 versus | Change from baseline in CDR-SB score |
| placebo on clinical progression as measured | |
| by the Clinical Dementia Rating-Sum of | |
| Boxes (CDR-SB) | |
| To characterize the relationship between tau | Actual values and changes from baseline in |
| PET standardized uptake value ratio (SUVR) | tau PET SUVR biomarkers, p217 + tau and |
| biomarkers (tau naive composite ROI and also | PACC-5total score |
| other ROIs), p217 + tau, and cognitive | |
| performance as measured by PACC-5 total | |
| score | |
| To assess the effect of ACI-35.030 on the | Change from baseline in brain tau burden, as |
| propagation and/or accumulation of tau | measured by tau PET in other regions of |
| pathology compared with placebo, as | interest (including but not limited to Braak I- |
| measured by tau PET in other regions of | VI ROIs, Connection Rank ROI, and New |
| interest | Tau Composite ROI Volume) |
| To evaluate changes in functional status | Change from baseline on the ADCSADL-PI |
| between participants treated with ACI-35.030 | |
| or placebo as measured by the ADCS | |
| Activities of Daily Living - Prevention | |
| Instrument (ADCSADL-PI) | |
| To investigate the effect of ACI-35.030 versus | Change from baseline in MBI-C score |
| placebo on the Mild Behavioral Impairment | |
| Checklist (MBI-C) | |
| To evaluate changes in quality of life between | Change from baseline on the QOL-AD |
| participants treated with ACI-35.030 or | |
| placebo as measured by the Quality of Life in | |
| AD (QOL-AD) | |
| To investigate the effect of ACI-35.030 versus | Change from baseline in EQ-5D-5L score |
| placebo on the European Quality of Life-5 | |
| Dimensions 5-levels (EQ-5D-5L) | |
| To evaluate differences in resource utilization | Change from baseline in resource utilization |
| between participants treated with ACI-35.030 | on RUD-Lite |
| or placebo as measured by Resource | |
| Utilization in Dementia Lite (RUD-Lite) | |
| To assess immunogenicity of ACI-35.030 in | IgG titers against enriched paired helical |
| serum | filaments (ePHF), p-tau and tau in serum |
| To assess overall safety and tolerability of | Treatment Emergent Adverse Events |
| ACI-35.030 versus placebo | (TEAEs) |
| | Reactogenicity |
| | Change from baseline in Vital signs (VS), |
| | Clinical Labs (chemistry, hematology, |
| | urinalysis), electrocardiogram (ECG), |
| | Columbia-Suicidality Severity Rating Scale |
| | (C-SSRS) |
| | Change in Magnetic Resonance Imaging |
| | (MRI) findings |
| Tertiary/Exploratory Objectives | Endpoints |
| To explore nature of the immune response | Measures of immune response may include: |
| induced by ACI-35.030 versus placebo | IgM titers against p-tau in serum, epitope |
| | mapping, antibodies against other |
| | immunotherapy components (eg, CpG, T50) |
| | and cytokines |
| To investigate the effects of ACI-35.030 | Delay (months) in cognitive decline based on |
| relative to placebo on delaying PACC-5 | the change in PACC-5 total score |
| cognitive decline measured in months over the | |
| treatment period | |
| To investigate effect of ACI-35.030 versus | To potentially include: p217 + tau, Aβ42/40, |
| placebo on blood and CSF AD biomarkers | p181 tau, total tau, NFL, GFAP, YKL-40, |
| | VILIP-1, TREM2 and other emerging AD |
| | biomarkers |
| To evaluate the effect of ACI-35.030 on | Change from baseline in hippocampal, whole |
| changes in brain volume as measured by | brain, and ventricular volume using MRI |
| volumetric MRI | |
| To explore functional capacity of | Functionality of immunotherapy-induced |
| immunotherapy-induced antibodies | antibodies as assessed via cellular in vitro |
| | assays |
| To assess epitope engagement in CSF | C-terminal tau fragment concentration in |
| | CSF |
| To assess immunogenicity of ACI-35.030 in | IgG titers against p-tau and tau in CSF |
| CSF | |
| To further explore the relationship between | Actual values/scores and changes from |
| biomarkers (imaging, blood, CSF, speech, | baseline for biomarkers (imaging, blood, |
| digital), immunogenicity, and clinical | CSF, speech, digital), immunogenicity, |
| outcomes, or other pharmacodynamic, | clinical outcome, or other pharmacodynamic, |
| efficacy or safety parameters as needed. | efficacy or safety parameters. |

This study will enroll approximately 498 participants, aged 55-75 years old, with preclinical AD, approximately 249 participants per treatment group.

The target population for this study will consist of participants, aged 55 to 75 years, who are cognitively unimpaired, with biomarker evidence of pathological phosphorylated tau protein (T+, evaluated first by plasma prescreen and confirmed tau pathology on tau PET). The target population can also have the biomarker evidence of amyloid pathology, e.g., elevated brain amyloid (A+ status). More specifically, to identify this population, the Lucent AD plasma p217+tau assay is being used to prescreen cognitively unimpaired individuals for evidence suggestive of elevated brain amyloid and pTau. The presence of pathologic tau as an eligibility criterion will be confirmed using tau PET (T+ status). Individuals meeting these criteria are considered to have preclinical AD.

Prescreening Eligibility Criteria: each potential participant must satisfy the following criteria:
55 to 75 years of age at the start of screening
A participant must be of non-childbearing potential.
No diagnosis of Alzheimer's Dementia or non-Alzheimer's Dementia or Mild Cognitive Impairment.
No history consistent with or known autosomal dominant AD (mutation identified in the family and/or participant).
Absence of any neurological, psychiatric, or medical conditions associated with a long-term risk of significant cognitive impairment or dementia (e.g., Parkinson's Disease).
No contraindications for MRI (e.g., certain prostheses and implants, claustrophobia, pacemaker).
Willingness to have an MRI and PET scan.
Must have a study partner who will accompany the participant to specified study visits.
Must sign a prescreening ICF indicating that participant understands the purpose of, and procedures required for the prescreening and is willing to participate in the prescreening.

Inclusion Criteria: each potential participant must satisfy all of the following criteria to be enrolled in the study:
Elevated brain tau pathology defined as Braak 3 ROI SUVR>1.1 on a screening tau PET scan, reviewed centrally by a qualified reader.
CDR global score of 0 at screening and baseline.
MMSE≥27 (with educational adjustment)
Able to read and write and with a minimum 5 years of formal education as reported by participant and study partner at screening.
Must have a study partner who will accompany the participant to specified study visits.

Exclusion Criteria: any potential participant who meets any of the following exclusion criteria for the study will be excluded from participating in the study:

(1) any of the following medical conditions:
MRI evidence of any brain disease or intracranial pathology other than potential very early signs of AD (e.g., mild hippocampal atrophy) or typical age-related changes (e.g., mild white matter hyperintensity on MRI)
History consistent with or known autosomal dominant AD (mutation identified in the family and/or participant)
Fulfills diagnostic criteria for Alzheimer's Dementia or non-Alzheimer's Dementia, including, but not limited to Frontotemporal Dementia (FTD), Diffuse Lewy Body Dementia (DLBD), Vascular Dementia (VAD), alcoholic dementia, Parkinson's dementia, Korsakov, Creutzfeldt-Jakob or other prion diseases, Posterior Cortical Atrophy
Diagnosis of Mild Cognitive Impairment (MCI)
Presence of any neurological, psychiatric, or medical conditions associated with a long-term risk of significant cognitive impairment or dementia including, but not limited to, pre-manifest Huntington's disease, multiple sclerosis, Parkinson's disease, Down's syndrome, active alcohol/drug abuse or major psychiatric disorders including, but not limited to, current diagnosis or history of schizophrenia, schizoaffective disorder, or bipolar affective disorder or current episode of major depressive disorder
Known allergies, hypersensitivity, or intolerance to ACI-35.030 or any components of ACI-35.030 or history of severe allergic reaction (eg, anaphylaxis) including but not limited to severe allergic reaction to previous vaccines and/or medications.

(2) Received an anti-amyloid or anti-tau therapy. If documented evidence can be provided the participant was treated with placebo in a clinical trial with these agents, participation would be allowed.

(3) Taken any disallowed therapies before the planned first dose of study intervention.

(4) Certain restrictions on prior/concomitant therapy or clinical Study Experience.

Pre-screening: The pre-screening phase will precede the screening period.

In order to most efficiently identify the study population and avoid unnecessary radiation exposure, a plasma biomarker will be used to prescreen cognitively unimpaired individuals for evidence suggestive of elevated brain tau (T+ status). The presence of pathological tau will be confirmed for all included participants using tau PET. p217+tau will be used only as a prescreen criterium to determine participants who are T+ for the purpose of continuing in screening with tau PET. During the pre-screening phase, eligibility for screening based on plasma p217+tau will be ascertained. The prescreening period will be approximately 4 weeks, but can be shorter or longer, depending on the availability of the plasma p217+tau result. Written pre-screening ICF is to be signed before any study procedure begins.

Participants meeting the pre-specified plasma p217+tau criteria can be screened for participation in the study and will be requested to visit the study site for the screening visit. Tau pathology stage on PET will be confirmed by a tau PET scan in screening.

Screening: After giving written informed consent for the study, participants will be screened over a period of up to 90 days to assess their eligibility for the study according to the inclusion and exclusion criteria. To allow for treatment of transient conditions, laboratory retesting, scheduling of MRI and tau PET, and optional lumbar puncture, and/or operational delays, the screening period of 90 days may be extended to 120 days with prior approval from the medical monitor on a case-by-case basis; if necessary, may be further extended up to 180 days with prior approval from medical monitor on a case-by-case basis. Participants that exceed 120 days may be required to repeat select study procedures. The study partner will have to come to the screening visit to sign the ICF and to complete clinical scales for the study partner.

Interventions: Eligible participants will be randomized in a 1:1 ratio after the screening period to receive either active treatment or placebo. The randomization will be balanced and stratified by geographical region and baseline tau burden. A sample size of 174 completers per group achieves 90% power to detect a group difference of 0.88 in change from baseline on PACC-5, assuming 2-sided alpha of 0.05 and SD of 2.53 (which translates to 40% slowing of cognitive decline compared to placebo). The ACI-35.030 drug product is supplied in 2 ml vials as a sterile liposome dispersion for intramuscular injection containing ACI-35.030 eq. 1200 μg/mL. The matching placebo is supplied as a final vialed product that must be stored at controlled temperatures and conditions as indicated on the product-specific labelling.

Participants are administered a dose of 900 μg of study intervention at Week 0 (Day 1), Week 8 (2 months), Week 24 (6 months) and every 26 weeks (˜6 months) until Week 180 (˜42 months) at the study site. The study intervention should be administered via an intramuscular injection in the deltoid muscle (upper arm). A schematic overview of the study's events can be found in FIG. 4.

Following dosing on Day 1 and during the entire double-blind treatment phase, treatment effects will be evaluated by means of imaging and fluid biomarkers, immunogenicity samples, cognitive and neuropsychological assessments, health outcome measures, and safety and tolerability. Study partners will have to accompany the visits at Week 0 (baseline), 24, 50, 76, 102, 128, 154, 180 and 206 to complete clinical scales for the study participants.

As of Week 102, the site will call the participants approximately 13 weeks after each study intervention administration to check on AEs and concomitant therapy.

After each study intervention administration, participants will remain under observation at the study site for a minimum of 30 minutes to monitor for presence of any acute reactions and solicited events, or longer if deemed necessary by the investigator. Any unsolicited and solicited local (injection site) or systemic AEs will be documented by the study site personnel following this observation period.

Evaluation: Efficacy assessments include cognitive decline as assessed by Preclinical Alzheimer's Cognitive Composite-5 (PACC-5), tau PET scans and other clinical outcomes [Mild Behavioral Impairment Checklist (MBI-C), Activities of Daily Living Prevention Instrument (ADCS-ADL-PI), CDR-GS and CDR-SB]. Immunogenicity and biomarker evaluation include assessment of blood- and CSF-based immunogenicity and AD biomarkers (e.g., p217+tau, total tau, AB42/40, NFL, GFAP, YKL-40, VILIP-1, TREM2). Impact on CSF phosphorylated C-terminal tau fragments may be evaluated. Digital biomarkers are used to evaluate longitudinal changes in neurocognitive functioning. Health outcomes are measured by the European Quality of Life-5 Dimensions 5-level (EQ-5D-5L) and Alzheimer's Disease Cooperative Study Quality of Life in Alzheimer's Disease (ADSC-QOL-AD). Resource utilization is measured via Resource Utilization in Dementia Lite (RUD Lite). IgG titers against ePHF, p-tau peptide and tau peptide in serum are measured. A pharmacogenomic blood sample is collected to allow for apolipoprotein E (APOE) allele status determination. Safety assessments include vital signs, ECG, blood and urine lab safety (including coagulation, thyroid function, vitamin B12, folic acid), C-SSRS, MRI, and physical and neurological examination.

It will be evident to one skilled in the art that the present disclosure is not limited to the foregoing illustrative examples, and that it can be embodied in other specific forms without departing from the essential attributes thereof: It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

SEQUENCE LISTING

SEQ ID NO: 1-phospho-Tau peptide (7.1)
GDRSGYS[pS]PG[pS]PG[pT]PGSRSRT
SEQ ID NO: 2-phospho-Tau peptide (T3.5)
VYK[pS]PVVSGDT[pS]PRHL
SEQ ID NO: 3-phospho-Tau peptide (22.1)
SSTGSIDMVD[pS]PQLA[pT]LA
SEQ ID NO: 4-Tau peptide (T3.6)
VYKSPVVSGDTSPRHL
SEQ ID NO: 5-phospho-Tau peptide
RENAKAKTDHGAEIVYK[pS]PVVSGDT[pS]PRHL
SEQ ID NO: 6-phospho-Tau peptide
RQEFEVMEDHAGT[pY]GL
SEQ ID NO: 7-phospho-Tau peptide
PGSRSR[pT]P[pS]LPTPPTR
SEQ ID NO: 8-phospho-Tau peptide
GYSSPG[pS]PG[pT]PGSRSR
SEQ ID NO: 9-phospho-Tau peptide
GDT[pS]PRHL[pS]NVSSTGSID
SEQ ID NO: 10-phospho-Tau peptide
PG[pS]PG[pT]PGSRSR[pT]P[pS]LP
SEQ ID NO: 11-phospho-Tau peptide
HL[pS]NVSSTGSID
SEQ ID NO: 12-phospho-Tau peptide
VSGDT[pS]PRHL
SEQ ID NO: 13-T50 T cell epitope
AKFVAAWTLKAAAVVRQYIKANSKFIGITELVVRFNNFTVSFWLRVPKVSASHLE-NH₂
SEQ ID NO: 14-T46 T cell epitope
AKFVAAWTLKAAAGSQYIKANSKFIGITELGSFNNFTVSFWLRVPKVSASHLEK(Pal)K(Pal)-
NH₂
SEQ ID NO: 15-T48 helper T cell epitope
AKFVAAWTLKAAAGSQYIKANSKFIGITELGSFNNFTVSFWLRVPKVSASHLEGSLINST
KIYSYFPSVISKVNQ-NH₂
SEQ ID NO: 16-T51 helper T cell epitope
AKFVAAWTLKAAARRQYIKANSKFIGITELRRFNNFTVSFWLRVPKVSASHLE-NH₂
SEQ ID NO: 17-T52 helper T cell epitope
AKFVAAWTLKAAARKQYIKANSKFIGITELRKFNNFTVSFWLRVPKVSASHLE-NH₂
SEQ ID NO: 18-CpG 2006 (also known as CpG 7909)
5′-tcgtcgttttgtcgttttgtcgtt-3′
wherein lower case means phosphorothioate (ps) internucleotide linkages
SEQ ID NO: 19-CpG 1018
5′-tgactgtgaacgttcgagatga-3′
wherein lower case means phosphorothioate internucleotide linkages
SEQ ID NO: 20-CpG2395
5′-tcgtcgttttcggcgcgcgccg-3′
wherein lower case means phosphorothioate internucleotide linkages
SEQ ID NO: 21-CpG2216
5′-ggGGGACGATCGTCgggggg-3′
wherein lower case means phosphorothioate internucleotide linkages and capital letters
means phosphodiester (po) linkages
SEQ ID NO: 22-CpG2336
5′-gggGACGACGTCGTGgggggg-3′,
wherein lower case means phosphorothioate internucleotide linkages and capital letters
means phosphodiester linkages
SEQ ID NO: 23-Pan DR epitope (PADRE) peptide
AKFVAAWTLKAAA
SEQ ID NO: 24-P2
QYIKANSKFIGITEL
SEQ ID NO: 25-P30
FNNFTVSFWLRVPKVSASHLE
SEQ ID NO: 26-TT₅₈₆₋₆₀₅
LINSTKIYSYFPSVISKVNQ
SEQ ID NO: 27-palmitoylated phospho-Tau peptide (palmitoylated 7.1)
K(pal)K(pal)GDRSGYS[pS]PG[pS]PG[pT]PGSRSRTK(pal)K(pal)
SEQ ID NO: 28-palmitoylated phospho-Tau peptide (T3, palmitoylated T3.5)
K(pal)K(pal)VYK[pS]PVVSGDT[pS]PRHLK(pal)K(pal)
SEQ ID NO: 29-palmitoylated phospho-Tau peptide (palmitoylated 22.1)
K(pal)K(pal)SSTGSIDMVD[pS]PQLA[pT]LAK(pal)K(pal)
SEQ ID NO: 30-palmitoylated Tau peptide
K(pal)K(pal)VYKSPVVSGDTSPRHLK(pal)K(pal)
SEQ ID NO: 31-palmitoylated phospho-Tau peptide
K(pal)K(pal)RENAKAKTDHGAEIVYK[pS]PVVSGDT[pS]PRHLK(pal)K(pal)
SEQ ID NO: 32-palmitoylated phospho-Tau peptide
K(pal)K(pal)RQEFEVMEDHAGT[pY]GLK(pal)K(pal)
SEQ ID NO: 33-palmitoylated phospho-Tau peptide
K(pal)K(pal)PGSRSR[pT]P[pS]LPTPPTRK(pal)K(pal)
SEQ ID NO: 34-palmitoylated phospho-Tau peptide
K(pal)K(pal)GYSSPG[pS]PG[pT]PGSRSRK(pal)K(pal)
SEQ ID NO: 35-palmitoylated phospho-Tau peptide
K(pal)K(pal)GDT[pS]PRHL[pS]NVSSTGSIDK(pal)K(pal)
SEQ ID NO: 36-palmitoylated phospho-Tau peptide
K(pal)K(pal)PG[pS]PG[pT]PGSRSR[pT]P[pS]LPK(pal)K(pal)
SEQ ID NO: 37-palmitoylated phospho-Tau peptide
K(pal)K(pal)HL[pS]NVSSTGSIDK(pal)K(pal)
SEQ ID NO: 38-palmitoylated phospho-Tau peptide
K(pal)K(pal)VSGDT[pS]PRHLK(pal)K(pal)
SEQ ID NO: 39-T50 without the C-terminal amide
AKFVAAWTLKAAAVVRQYIKANSKFIGITELVVRFNNFTVSFWLRVPKVSASHLE
SEQ ID NO: 40-T46 without the -Lys(Pal)-Lys(Pal)-NH₂ at the C-terminal
AKFVAAWTLKAAAGSQYIKANSKFIGITELGSFNNFTVSFWLRVPKVSASHLE
SEQ ID NO: 41-T48 without the C-terminal amide
AKFVAAWTLKAAAGSQYIKANSKFIGITELGSFNNFTVSFWLRVPKVSASHLEGSLINST
KIYSYFPSVISKVNQ
SEQ ID NO: 42-T51 without the C-terminal amide
AKFVAAWTLKAAARRQYIKANSKFIGITELRRFNNFTVSFWLRVPKVSASHLE
SEQ ID NO: 43-T52 without the C-terminal amide
AKFVAAWTLKAAARKQYIKANSKFIGITELRKFNNFTVSFWLRVPKVSASHLE
SEQ ID NO: 44-T57
AKFVAAWTLKAAAVVRQYIKANSKFIGITELVVRFNNFTVSFWLRVPKVSASHLE-
K(Pal)K(Pal)-NH2
SEQ ID NO: 45-biotinylated phosphorylated tau peptide (T3.30)
Biotin-LC linker (Ahx)-GVYK[pS]PVVSGDT[pS]PRHL-NH2
SEQ ID NO: 46-biotinylated non-phosphorylated tau peptide (T3.56)
Biotin-LC linker (Ahx)-GVYKSPVVSGDTSPRHL-NH2
SEQ ID NO: 47-biotinylated phosphorylated tau peptide (T3.85)
Biotin-LC linker (Ahx)-VYK[pS]PVVSGDT[pS]PRHLS-NH2
SEQ ID NO: 48-biotinylated non-phosphorylated tau peptide (T3.86)
Biotin-LC linker (Ahx)-VYKSPVVSGDTSPRHLS-NH2

We claim:

1. A method of treating preclinical Alzheimer's disease in a human subject in need thereof, comprising intramuscularly administering an effective amount of a liposome to the human subject, wherein the liposome comprises:
(i) a tau peptide having the amino acid sequence of SEQ ID NO: 2, wherein the tau peptide is presented on the surface of the liposome;
(ii) a helper T cell epitope comprising at least one amino acid sequence selected from the group consisting of: SEQ ID NOs: 23, 24, 25, and 26;
(ii) a lipidated CpG oligonucleotide, wherein the CpG oligonucleotide comprises one or more phosphorothioate internucleotide linkages, and wherein the CpG oligonucleotide is covalently linked to at least one cholesterol via a linker; and
(iv) monophosphoryl lipid A (MPLA),
wherein the human subject has a tau pathology and no cognitive impairment.

2. The method of claim 1, wherein the human subject has a tau pathology as measured by an elevated plasma level of p-tau217, p-tau181, and/or p-tau231, and/or by a tau positron emission tomography (PET) scan at the initial administration of the effective amount of the liposome.

3. The method of claim 2, wherein the human subject has no diagnosis of Alzheimer's Dementia or non-Alzheimer's Dementia or Mild Cognitive Impairment, and has at least one of (i) an elevated brain tau pathology defined as Braak III region of interest standardized uptake value ratio (Braak 3 ROI SUVR)>1.1 on a screening tau PET scan, (ii) a Clinical Dementia Rating (CDR) global score of 0, and/or (iii) a Mini-Mental State Examination (MMSE) score of 25 or more, at the initial administration of the effective amount of the liposome.

4. The method of claim 1, wherein the human subject is 50-75 years of age at the initial administration of the effective amount of the liposome.

5. The method of claim 1, wherein the effective amount of the liposome comprises 300 μg to 1800 μg per dose of the tau peptide.

6. The method of claim 1, wherein the effective amount of the liposome comprises 900 μg per dose of the tau peptide.

7. The method of claim 1, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

8. A method of treating preclinical Alzheimer's disease in a human subject in need thereof, comprising intramuscularly administering an effective amount of a liposome to the human subject once every 6 months for a period of at least 1 year, wherein the liposome comprises:
(i) a tau peptide having the amino acid sequence of SEQ ID NO: 2, wherein the tau peptide is presented on the surface of the liposome;
(ii) a helper T cell epitope comprising at least one amino acid sequence selected from the group consisting of: SEQ ID NOs: 23, 24, 25, and 26;
(iii) a lipidated CpG oligonucleotide, wherein the CpG oligonucleotide comprises one or more phosphorothioate internucleotide linkages, and wherein the CpG oligonucleotide is covalently linked to at least one cholesterol via a linker, and
(iv) monophosphoryl lipid A (MPLA);
wherein the human subject has a tau pathology and no cognitive impairment.

9. The method of claim 8, wherein the human subject has a tau pathology as measured by an elevated plasma level of p-tau217, p-tau181, and/or p-tau231, and/or by a tau positron emission tomography (PET) scan at the initial administration of the effective amount of the liposome.

10. The method of claim 9, wherein the human subject has no diagnosis of Alzheimer's Dementia or non-Alzheimer's Dementia or Mild Cognitive Impairment, and has at least one of (i) an elevated brain tau pathology defined as Braak III region of interest standardized uptake value ratio (Braak 3 ROI SUVR)>1.1 on a screening tau PET scan, (ii) a Clinical Dementia Rating (CDR) global score of 0, and/or (iii) a Mini-Mental State Examination (MMSE) score of 25 or more, at the initial administration of the effective amount of the liposome.

11. The method of claim 8, wherein the human subject is 50-75 years of age at the initial administration of the effective amount of the liposome.

12. The method of claim 8, wherein the effective amount of the liposome comprises 300 μg to 1800 μg per dose of the tau peptide.

13. The method of claim 12, wherein the effective amount of the liposome comprises 900 μg per dose of the tau peptide.

14. The method of claim 8, further comprising:
(a) intramuscularly administering the effective amount of the liposome to the human subject 24 weeks before initiating the once every 6 months administrations; and
(b) intramuscularly administering the effective amount of the liposome to the human subject 16 weeks before initiating the once every 6 months administrations.

15. The method of claim 8, wherein the effective amount of the liposome is administered to the human subject once every 6 months for a period of at least 1.5 years, at least 2 years, at least 2.5 years, at least 3 years, at least 3.5 years or at least 4 years.

16. The method of claim 8, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

17. A method of treating preclinical Alzheimer's disease in a human subject, comprising:
(a) intramuscularly administering an effective amount of a liposome to the human subject at week 0 of the treatment;
(b) intramuscularly administering the effective amount of the liposome to the human subject at week 8 of the treatment; and
(c) intramuscularly administering the effective amount of the liposome to the human subject once every 6 months for a period of at least 1 year, wherein the period starts at week 24 of the treatment,
wherein the liposome comprises:
(i) a tau peptide having the amino acid sequence of SEQ ID NO: 28, wherein the tau peptide is presented on the surface of the liposome;
(ii) a helper T cell epitope having an amino acid sequence selected from the group consisting of SEQ ID NOs. 39, 40, 41, 42, and 43;
(iii) a lipidated CpG oligonucleotide having a nucleotide sequence selected from the group consisting of SEQ ID NO: 18 to SEQ ID NO:22, wherein the CpG oligonucleotide is covalently linked to at least one cholesterol via a linker; and
(iv) monophosphoryl lipid A (MPLA),
wherein the human subject has a tau pathology and no cognitive impairment.

18. The method of claim 17, wherein the human subject has a tau pathology as measured by an elevated plasma level of p-tau217, p-tau181, and/or p-tau231, and/or by a tau PET scan, at the initial administration of the effective amount of the liposome.

19. The method of claim 18, wherein at week 0, the human subject has no diagnosis of Alzheimer's Dementia or non-Alzheimer's Dementia or Mild Cognitive Impairment, and has at least one of (i) an elevated brain tau pathology defined as Braak III region of interest standardized uptake value ratio (Braak 3 ROI SUVR)>1.1 on a screening tau PET scan, (ii) a Clinical Dementia Rating (CDR) global score of 0, and/or (iii) a Mini-Mental State Examination (MMSE) score of 27 or more, at the initial administration of the effective amount of the liposome.

20. The method of claim 17, wherein the human subject is 50-75 years of age at week 0.

21. The method of claim 20, wherein the effective amount of the liposome comprises 300 μg to 1800 μg per dose of the tau peptide.

22. The method of claim 21, wherein the effective amount of the liposome comprises 900 μg per dose of the tau peptide.

23. The method of claim 17, wherein the effective amount of the liposome is administered to the human subject once every 6 months for a period of at least 1.5 years, at least 2 years, at least 2.5 years, at least 3 years, at least 3.5 years or at least 4 years.

24. The method of claim 17, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).

25. A method of treating preclinical Alzheimer's disease in a human subject, comprising:
(a) intramuscularly administering an effective amount of a liposome to the human subject at week 0 of the treatment;
(b) intramuscularly administering the effective amount of the liposome to the human subject at week 8 of the treatment; and
(c) intramuscularly administering the effective amount of the liposome to the human subject once every 6 months for a period of at least 1 year, wherein the period starts at week 24 of the treatment,
wherein the liposome comprises:
(i) a tau peptide comprising the amino acid sequence of SEQ ID NO: 28, wherein the tau peptide is presented on the surface of the liposome;
(ii) a helper T cell epitope having an amino acid sequence selected from the group consisting of SEQ ID NOs: 13, 14 or 15;
(iii) a lipidated CpG oligonucleotide having the nucleotide sequence selected from the group consisting of SEQ ID NO: 18, wherein the CpG oligonucleotide is covalently linked to at least one cholesterol via a linker; and
(iv) monophosphoryl lipid A (MPLA),
wherein the human subject is 50-75 years of age and has a tau pathology as measured by an elevated plasma level of p-tau217 and a tau PET scan at week 0 and has no cognitive impairment.

26. The method of claim 25, wherein at week 0, the human subject has no diagnosis of Alzheimer's Dementia or non-Alzheimer's Dementia or Mild Cognitive Impairment, and has (i) an elevated brain tau pathology defined as Braak III region of interest standardized uptake value ratio (Braak 3 ROI SUVR)>1.1 on a screening tau PET scan, (ii) a CDR global score of 0, and (iii) a MMSE score of 27 or more.

27. The method of claim 25, wherein the effective amount of the liposome comprises 300 μg to 1800 μg per dose of the tau peptide.

28. The method of claim 27, wherein the effective amount of the liposome comprises 900 μg per dose of the tau peptide.

29. The method of claim 25, wherein the effective amount of the liposome is administered to the human subject once every 6 months for a period of at least 1.5 years, at least 2 years, at least 2.5 years, at least 3 years, at least 3.5 years or at least 4 years.

30. The method of claim 25, wherein the treatment results in prevention and/or delay of the onset of cognitive impairment related to Alzheimer's disease in the human subject as measured by at least one of:
a. Preclinical Alzheimer's Cognitive Composite-5 (PACC-5) total score;
b. Preclinical Alzheimer's Cognitive Composite-5 (PACC-5) individual domain scores;
c. tau PET in the Tau Naive Composite region of interest, or other regions of interest;
d. the Clinical Dementia Rating-Global Score (CDR-GS), or the Clinical Dementia Rating-Sum of Boxes (CDR-SB); or
e. the ADCS Activities of Daily Living-Prevention Instrument (ADCSADL-PI), the Mild Behavioral Impairment Checklist (MBI-C), the Quality of Life in AD (QOL-AD), the European Quality of Life-5 Dimensions 5-levels (EQ-5D-5L), or the Resource Utilization in Dementia Lite (RUD-Lite).
